(19) United States
(12) Patent Application Publication
(10) Pub. No.: US 2022/0233781 A1
(43) Pub. Date: Jul. 28, 2022

(54) INJECTION DEVICE WITH DOSAGE MONITORING

(71) Applicant: Dali Medical Devices Ltd, Yavne (IL)

(72) Inventors: Dmitri Sarkorov, Rosh Ha'ayin (IL); David Daily, Herzliya (IL); Lior Raday, Kibbutz Bror-Hail (IL); Gad Lewkonya, Neve Mivtach (IL); Eyall Abir, Petach Tikva (IL)

(21) Appl. No.: 17/615,588

(22) PCT Filed: Jun. 24, 2020
(86) PCT No.: PCT/IL2020/050704

§ 371 Date: Dec. 1, 2021

Related U.S. Application Data
(60) Provisional application No. 62/916,815, filed on Oct. 18, 2019.

(30) Foreign Application Priority Data
Jun. 24, 2019 (IL) 267610

(51) Int. Cl. A61M 5/315; A61M 5/31

(52) U.S. Cl. CPC A61M 5/31568; A61M 5/3158; A61M 5/31593; A61M 2205/50; A61M 5/31511; A61M 2005/3139; A61M 2005/3152; A61M 5/3137

(57) ABSTRACT

Injection device and method for monitoring injection information relating to an injection. A syringe of the device includes a syringe chamber containing an injectant substance. A plunger of the device includes a plunger rod axially displaceable within the syringe chamber. At least one element of the is configured to undergo a corresponding element motion linked to a distal displacement of the plunger rod. A sensor of an electronics unit of the device obtains detection readings relating to the corresponding element motion. An injection is performed by depressing the plunger rod in a distal direction, compelling a corresponding element motion detected by the sensor, and compelling the injectant substance to be injected through a distal end of the syringe chamber. The detection readings relating to the corresponding element motion of the element is processed to determine injection information including at least the volume of injectant substance injected.

### FIELD OF THE INVENTION

The present invention generally relates to hypodermic needles and syringes, and particularly to syringes for measuring and monitoring an injectant dosage.

### BACKGROUND OF THE INVENTION

Injection devices are widely used for administering injections, such as for injecting a medication or drug into the body of a patient. One of the most common types of injection devices is a syringe. A syringe generally consists of a plunger, tightly fitted within a cylindrical barrel or chamber, and a hypodermic needle fitted at the distal end of the syringe. The plunger is linearly advanceable within the syringe chamber, which contains an injectant substance. The injection is implemented by piercing the skin with the needle at a selected body region and then manually depressing the plunger, which forces the injectant substance to be ejected through the needle aperture. The syringe or the needle is typically intended for single use (disposable), but is sometimes designated for repeated usage. Syringes are frequently utilized in clinical medicine to administer drugs or medications, to deliver fluids into the bloodstream for infusions or intravenous therapy, to apply compounds (e.g., such as adhesives or lubricants), or to extract and measure fluids (e.g., blood samples). Syringes and injection devices come in a wide variety of different types and categories, including safety syringes, injection pens, auto-injector devices, and insulin pumps. For example, a safety syringe may incorporate a retraction mechanism to retract the needle inside the syringe chamber after completion of the injection, in order to preclude contamination and prevent potential injury from the exposed needle.

Certain medical treatments involving syringe injections must be performed by a clinician or certified medical staff and requires the patient to be hospitalized. Such hospitalizations can be expensive and time-consuming. A patient may alternatively visit an outpatient clinic to receive the injection, which can also be troublesome and inconvenient. Self-administering an injection can be difficult and is generally considered unreliable, and can result in improper injections and medical complications. For example, it may be especially difficult to measure and monitor a precise dosage of the medication or substance that needs to be injected. When conducting medical research involving periodic injections of a subject patient, self-administered injections is considered to compromise the veracity of the usage.

Various devices and methods for measuring injected dosages are known in the art. Many of these devices have limitations, such as lack of reliability, complicated designs, and costly manufacturing expenses.

U.S. Pat. No. 6,352,523 to Brown et al., entitled: “Capacitance-based dose measurements in syringes”, is directed to an apparatus and method for capacitively measuring insulin quantities in a syringe in an integrated insulin dose recorder/blood glucose meter. The syringe is placed in a holder before and after the administration of the dose. Capacitor electrodes may be situated within the syringe and/or outside the syringe in various geometries. Liquid quantities in the syringe are determined by comparing capacitive response patterns of the syringe with calibration data stored in the device. Dose histories are downloaded to a patient computer for transfer to a clinician's computer.

U.S. Pat. No. 6,743,202 to Hirschman et al., entitled: “Encoding of syringe information”, is directed to an apparatus and method for injecting fluid into a patient in which syringe information relevant to the injection procedure is encoded and shared with an injector. The encoded syringe information is readable by a detection circuit in the injector, which may include electrically conductive contact readout members. The encoded syringe information is stored by a storage system, which may include electrically conductive code contact members. The syringe information may be conveyed to the detector when contact is made between the storage system and the detector.

European Patent No. 1,827,537 to Enggaard et al., entitled: “Medication delivery system with a detector for providing a signal indicative of an amount of an ejected dose of drug”, is directed to a medication delivery system for identified the amount of an ejected dose. The system includes a movable part adapted to move relative to a stationary part, at least two conductors arranged such that an electrical characteristic is defined by the mutual position or relative movement of the two parts, and a detector for detecting a change of the electrical characteristic. The parts are stationary relative to each other during the does setting, and moved relative to each other during the dose ejections, such that the detector provides a signal indicative of the actual amount of the ejected dose.

U.S. Pat. No. 9,586,009 to Butler et al., entitled: “Drug delivery device”, is directed to a pen-type injection device that provides injection of medicinal products from a multi-dose cartridge where a user may set the dose. The device includes a cylindrical member rotatably supported inside a housing. The outer surface of the cylindrical member is provided with at least first and second tracks together forming an encoder. Each track includes conductive segments and non-conductive segments. First and second groups of contacts are configured to engage the first and second tracks respectively at predetermined intervals along the length of the track.

The aforementioned publications generally relate to electromechanical devices which generate an electrical signal that reflects the dosage of injected material. Other approaches for measuring injectant dosages are also known in the art. For example, U.S. Pat. No. 9,255,830 to Whalley et al., entitled: “Dose measurement system and method”, discloses an optical-based technique for measuring the dose remaining in a drug delivery device. A plurality of light sources is disposed in an apparatus and configured to emit electromagnetic radiation toward a container. A plurality of sensors optically couplable to the light sources are located in the apparatus and configured to detect the emitted electromagnetic radiation. A processing unit is configured to receive data representing the detected electromagnetic information from the sensors and convert the received data into a representative signature. The signature may be representative of a drug volume in the drug delivery device. The processing unit may include a memory, such as an RFID chip, configured to store information, such as the remaining dose.

Another relevant publication is U.S. Patent Application No. 2019/0054251 to Pieronek et al., entitled: “Medicine delivery device”, directed to techniques for sensing a dosage setting at a medicine delivery device. The device includes a container configured to store medicine, and a movable component configured to set a dosage of medicine to be dispensed. A dispensing mechanism is configured to deliver the dosage of the medicine, and one or more sensors are configured to generate data samples related to a movement of the movable component. A processor determined based on the data samples a direction and a distance of at least one movement component, determines based on the direction and distance a dosage of medicine set by the movable component, compares the dosage against a preset threshold, and performs one or more actions related to the delivery of the dosage based on the comparison. Information may also be communicated remotely from the medicine delivery device, such as to a patient's mobile device.

### SUMMARY OF THE INVENTION

In accordance with one aspect of the present invention, there is thus provided an injection device. The device has a distal end and a proximal end. The device includes a syringe, which includes a syringe chamber containing an injectant substance. The device further includes a plunger, which includes a plunger rod axially displaceable within the syringe chamber. The device further includes at least one element, configured to undergo a corresponding element motion linked to a distal displacement of the plunger rod. The device further includes an electronics unit, including at least one sensor, configured to obtain detection readings relating to the corresponding element motion of the element. An injection is performed by depressing the plunger rod in a distal direction, compelling a corresponding element motion detected by the sensor, and compelling the injectant substance to be injected through a distal end of the syringe chamber. The detection readings relating to the corresponding element motion of the element is processed to determine injection information including at least the volume of injectant substance injected. The element may include a rotor, which includes a rotor rod, disposed concentrically within the plunger rod, and a rotor head, at a proximal end of the rotor rod. The rotor head is configured to rotate in accordance with a distal advancement of the plunger rod, such that the corresponding element motion is a rotational motion of the rotor head, where the sensor is configured to detect the rotational motion of the rotor head. The rotor rod may further include a helical screw thread, and the injection device may further include at least one guiding pin, configured to engage the screw thread, so as to turn the rotor rod, producing the rotational motion of the rotor head. The guiding pin may be disposed on a ring element, at least partially encircling the plunger rod, the guiding pin extending radially inward from the ring element, where the guiding pin engages the screw thread through a plunger rod slot, extending axially along the plunger rod, such that the guiding pin is restricted to linear axial motion with respect to the plunger rod by the plunger rod slot, preventing rotation of the ring element with respect to the plunger rod. The rotor head may include a plurality of rotor head apertures, where the sensor is configured to detect a sequential passage of the rotor head apertures during the rotational motion of the rotor head. The element may include a rotor, which includes a rotor head, at least partially encircling the plunger rod, where the rotor head is configured to rotate in accordance with a distal advancement of the plunger rod, such that the corresponding element motion is a rotational motion of the rotor head, where the sensor is configured to detect the rotational motion of the rotor head during the distal advancement of the plunger rod. The plunger rod may include a helical screw thread, where the rotor head further includes at least one guiding pin, extending radially inward from an inner wall of the rotor head, the guiding pin configured to engage the screw thread, so as to turn the rotor, producing the rotational motion of the rotor head. The injection device may further include a gripping unit, which includes a gripping unit opening, such that the plunger rod extends through the gripping unit opening, shaped with at least one edge aligned with an edge of the plunger rod, so as to prevent a rotation of the plunger rod relative to the gripping unit. The rotor head may include a plurality of rotor head apertures, where the sensor is configured to detect a sequential passage of the rotor head apertures during the rotational motion of the rotor head. The element may include a plurality of teeth, extending axially along a portion of the plunger rod, each tooth of the teeth including a respective tooth protrusion, protruding radially outwards from the plunger rod, and a respective tooth indentation, protruding radially inwards from the plunger rod, where the corresponding element motion is an axial motion of the teeth, and where the sensor is configured to detect the sequential passage of the teeth during the distal advancement of the plunger rod. The injection device may further include a gripping unit, comprising a gripping unit opening, such that the plunger rod extends through the gripping unit opening, shaped with at least one edge aligned with an edge of the plunger rod, so as to prevent a rotation of the plunger rod relative to the gripping unit. The element may include a rack, positioned within the plunger rod, the rack including a plurality of teeth, extending axially along a portion of the rack, each tooth of the teeth comprising a respective tooth protrusion, projecting radially through an axial slot of the plunger rod when the rack is positioned within the plunger rod, where the corresponding element motion is a sequential axial motion of the rack, and where the sensor is configured to detect the sequential axial motion of the rack during the distal advancement of the plunger rod. The injection device may further include a substantially flexible ring, at least partially encircling the plunger rod, the ring including at least one ring tooth extending radially inward from the ring, the ring tooth configured to engage with a rack tooth of the rack through the axial slot of the plunger rod, such that the ring is prevented from rotation with respect to the plunger rod. The ring may include at least one flexible portion, configured to selectively deform radially, where during the distal advancement of the plunger rod, the rack is restricted from distal displacement by the ring tooth, resulting in a proximal counterforce applied by the rack, initiating a cyclical activation of the sensor and a cyclical radial expansion of the ring, allowing the distal advancement of a respective rack tooth through the ring, and detection of the rack tooth distal advancement by the sensor for each cycle. The electronics unit may further include a transmitting antenna, configured to transmit the detection readings to a remote location. The electronics unit may further include an indicator, configured to provide an indication of at least one injection state relating to the injection.

In accordance with another aspect of the present invention, there is thus provided a method for monitoring injection information relating to an injection. The method includes the procedure of providing an injection device, the device having a distal end and a proximal end, the device including a syringe, which includes a syringe chamber containing an injectant substance, the device further including a plunger, which includes a plunger rod axially displaceable within the syringe chamber, the device further including at least one element, configured to undergo a corresponding element motion linked to a distal displacement of the plunger rod, the device further including an electronics unit, including at least one sensor, configured to obtain detection readings relating to the corresponding element motion of the element. The method further includes the procedure of performing an injection by depressing the plunger rod in a distal direction, compelling a corresponding element motion detected by the sensor, and compelling the injectant substance to be injected through a distal end of the syringe chamber. The method further includes the procedure of processing the detection readings relating to the corresponding element motion of the element to determine injection information including at least the volume of injectant substance injected. The element may include a rotor, which includes a rotor rod, disposed concentrically within the plunger rod, and a rotor head, at a proximal end of the rotor rod. The rotor head is configured to rotate in accordance with a distal advancement of the plunger rod, such that the corresponding element motion is a rotational motion of the rotor head, where the sensor is configured to detect the rotational motion of the rotor head. The rotor rod may further include a helical screw thread, and the injection device may further include at least one guiding pin, configured to engage the screw thread, so as to turn the rotor rod, producing the rotational motion of the rotor head. The guiding pin may be disposed on a ring element, at least partially encircling the plunger rod, the guiding pin extending radially inward from the ring element, where the guiding pin engages the screw thread through a plunger rod slot, extending axially along the plunger rod, such that the guiding pin is restricted to linear axial motion with respect to the plunger rod by the plunger rod slot, preventing rotation of the ring element with respect to the plunger rod. The rotor head may include a plurality of rotor head apertures, where the sensor is configured to detect a sequential passage of the rotor head apertures during the rotational motion of the rotor head. The element may include a rotor, which includes a rotor head, at least partially encircling the plunger rod, where the rotor head is configured to rotate in accordance with a distal advancement of the plunger rod, such that the corresponding element motion is a rotational motion of the rotor head, where the sensor is configured to detect the rotational motion of the rotor head during the distal advancement of the plunger rod. The plunger rod may include a helical screw thread, where the rotor head further includes at least one guiding pin, extending radially inward from an inner wall of the rotor head, the guiding pin configured to engage the screw thread, so as to turn the rotor, producing the rotational motion of the rotor head. The injection device may further include a gripping unit, which includes a gripping unit opening, such that the plunger rod extends through the gripping unit opening, shaped with at least one edge aligned with an edge of the plunger rod, so as to prevent a rotation of the plunger rod relative to the gripping unit. The rotor head may include a plurality of rotor head apertures, where the sensor is configured to detect a sequential passage of the rotor head apertures during the rotational motion of the rotor head. The element may include a plurality of teeth, extending axially along a portion of the plunger rod, each tooth of the teeth including a respective tooth protrusion, protruding radially outwards from the plunger rod, and a respective tooth indentation, protruding radially inwards from the plunger rod, where the corresponding element motion is an axial motion of the teeth, and where the sensor is configured to detect the sequential passage of the teeth during the distal advancement of the plunger rod. The injection device may further include a gripping unit, comprising a gripping unit opening, such that the plunger rod extends through the gripping unit opening, shaped with at least one edge aligned with an edge of the plunger rod, so as to prevent a rotation of the plunger rod relative to the gripping unit. The element may include a rack, positioned within the plunger rod, the rack including a plurality of teeth, extending axially along a portion of the rack, each tooth of the teeth comprising a respective tooth protrusion, projecting radially through an axial slot of the plunger rod when the rack is positioned within the plunger rod, where the corresponding element motion is a sequential axial motion of the rack, and where the sensor is configured to detect the sequential axial motion of the rack during the distal advancement of the plunger rod. The injection device may further include a substantially flexible ring, at least partially encircling the plunger rod, the ring including at least one ring tooth extending radially inward from the ring, the ring tooth configured to engage with a rack tooth of the rack through the axial slot of the plunger rod, such that the ring is prevented from rotation with respect to the plunger rod. The ring may include at least one flexible portion, configured to selectively deform radially, where during the distal advancement of the plunger rod, the rack is restricted from distal displacement by the ring tooth, resulting in a proximal counterforce applied by the rack, initiating a cyclical activation of the sensor and a cyclical radial expansion of the ring, allowing the distal advancement of a respective rack tooth through the ring, and detection of the rack tooth distal advancement by the sensor for each cycle. The method may further include the procedure of transmitting the detection readings to a remote location with a transmitting antenna of the electronics unit. The method may further include the procedure of providing an indication of at least one injection state relating to the injection with an indicator of the electronics unit.

### BRIEF DESCRIPTION OF THE DRAWINGS

The present invention will be understood and appreciated more fully from the following detailed description taken in conjunction with the drawings in which:

FIG. 1 is a perspective view illustration of an injection device in an assembled configuration, constructed and operative in accordance with a first embodiment of the present invention;

FIG. 2 is an exploded perspective view illustration of the injection device of FIG. 1, constructed and operative in accordance with a first embodiment of the present invention;

FIG. 3 is a perspective view illustration of the gripping unit of the injection device of FIG. 1, constructed and operative in accordance with a first embodiment of the present invention;

FIG. 4 is a perspective view illustration of the ring element of the injection device of FIG. 1, constructed and operative in accordance with a first embodiment of the present invention;

FIG. 5A is a perspective view illustration of the plunger of the injection device of FIG. 1, constructed and operative in accordance with a first embodiment of the present invention;

FIG. 5B is a lateral sectional view illustration of the plunger of FIG. 5A;

FIG. 5C is a rotated lateral sectional view illustration of the plunger view of FIG. 5B;

FIG. 5D is a proximal view illustration of the plunger of FIG. 5A;

FIG. 6A is a perspective view illustration of the rotor of the injection device of FIG. 1, constructed and operative in accordance with a first embodiment of the present invention;

FIG. 6B is a lateral view illustration of the rotor of FIG. 6A;

FIG. 7A is a perspective view illustration of the cap of the injection device of FIG. 1, depicting a proximal surface thereof, constructed and operative in accordance with a first embodiment of the present invention;

FIG. 7B is a perspective view illustration of the cap of FIG. 7A, depicting a distal surface thereof;

FIG. 8A is a perspective view illustration of the electrical board of the injection device of FIG. 1, depicting a distal surface thereof, constructed and operative in accordance with a first embodiment of the present invention;

FIG. 8B is a perspective view illustration of the electrical board of FIG. 8A, depicting a proximal surface thereof;

FIG. 9A is an orthographic view illustration of the injection device of FIG. 1 in an initial operational state, constructed and operative in accordance with a first embodiment of the present invention;

FIG. 9B is a rotated orthographic view illustration of the injection device of FIG. 9A;

FIG. 9C is a sectional view illustration of the injection device of FIG. 9B;

FIG. 10 is a perspective view illustration of the rotor and ring element of the injection device of FIG. 1, when in an initial operational state, constructed and operative in accordance with a first embodiment of the present invention;

FIG. 11 is an orthographic view illustration of the injection device of FIG. 1) prior to the needle insertion with the rigid needle shield (RNS) removed, constructed and operative in accordance with a first embodiment of the present invention;

FIG. 12A is an orthographic view illustration of the injection device of FIG. 1 at a later operational state with the plunger rod fully depressed, constructed and operative in accordance with a first embodiment of the present invention;

FIG. 12B is an orthographic view illustration of the injection device of FIG. 12A at a later operational state with the plunger rod fully depressed;

FIG. 12C is a sectional view illustration of the injection device of FIG. 12B;

FIG. 13 is a perspective view illustration of an injection device in an assembled configuration, constructed and operative in accordance with a second embodiment of the present invention;

FIG. 14 is an exploded perspective view illustration of the injection device of FIG. 13, constructed and operative in accordance with a second embodiment of the present invention;

FIG. 15A is a perspective view illustration of the gripping unit of the injection device of FIG. 13, constructed and operative in accordance with a second embodiment of the present invention;

FIG. 15B is a rotated view illustration of the gripping unit view of FIG. 15A;

FIG. 16 is a perspective view illustration of the ring element of the injection device of FIG. 13, constructed and operative in accordance with a second embodiment of the present invention;

FIG. 17A is a perspective view illustration of the cap of the injection device of FIG. 13, depicting a distal surface thereof, constructed and operative in accordance with a second embodiment of the present invention;

FIG. 17B is a perspective view illustration of the cap of FIG. 17A, depicting a proximal surface thereof;

FIG. 18A is a perspective view illustration of the electrical board of the injection device of FIG. 13, depicting a distal surface thereof, constructed and operative in accordance with a second embodiment of the present invention;

FIG. 18B is a perspective view illustration of the electrical board of FIG. 18A, depicted a proximal surface thereof;

FIG. 19 is a perspective view illustration of the plunger of the injection device of FIG. 13, constructed and operative in accordance with a second embodiment of the present invention;

FIG. 20 is a perspective view illustration of the plunger with ring element of the injection device of FIG. 13, constructed and operative in accordance with a second embodiment of the present invention;

FIG. 21A is an orthographic distal view illustration of the rotor and the sensor of the injection device of FIG. 13, constructed and operative in accordance with a second embodiment of the present invention;

FIG. 21B is an orthographic lateral view illustration of the rotor and the sensor of FIG. 21A;

FIG. 21C is an orthographic proximal view illustration of the rotor and the sensor of FIG. 21A;

FIG. 22A is an orthographic view illustration of the injection device of FIG. 13, when in an initial operational state, constructed and operative in accordance with a second embodiment of the present invention;

FIG. 22B is a rotated orthographic view illustration of the injection device of FIG. 22A;

FIG. 22C is a sectional orthographic view illustration of the injection device of FIG. 22B;

FIG. 23 is an orthographic view illustration of the injection device of FIG. 13, prior to the needle insertion with the rigid needle shield (RNS) removed, constructed and operative in accordance with a second embodiment of the present invention;

FIG. 24A is an orthographic view illustration of the injection device of FIG. 13 at a later operational state with the plunger rod fully depressed, constructed and operative in accordance with a second embodiment of the present invention;

FIG. 24B is a sectional orthographic view illustration of the injection device of FIG. 24A;

FIG. 25 is a perspective view illustration of an injection device in an assembled configuration, constructed and operative in accordance with a third embodiment of the present invention;

FIG. 26 is an exploded perspective view illustration of the injection device of FIG. 25, constructed and operative in accordance with a third embodiment of the present invention;

FIG. 27A is a perspective proximal view illustration of the gripping unit of the injection device of FIG. 25, constructed and operative in accordance with a first embodiment of the present invention;

FIG. 27B is a perspective distal view illustration of the gripping unit of FIG. 27A;

FIG. 28A is a perspective view illustration of the cap of the injection device of FIG. 25, depicting a proximal surface thereof, constructed and operative in accordance with a third embodiment of the present invention;

FIG. 28B is a perspective view illustration of the cap of FIG. 28A, depicting a distal surface thereof;

FIG. 29A is a perspective view illustration of the electrical board of the injection device of FIG. 25, depicting a proximal surface thereof, constructed and operative in accordance with a third embodiment of the present invention;

FIG. 29B is a perspective view illustration of the electrical board of FIG. 29A, depicting a distal surface thereof;

FIG. 30 is a perspective view illustration of the plunger of the injection device of FIG. 25, constructed and operative in accordance with a third embodiment of the present invention;

FIG. 31A is an orthographic view illustration of the plunger and the sensor of the injection device of FIG. 25, constructed and operative in accordance with a third embodiment of the present invention;

FIG. 31B is an orthographic view illustration of the plunger and sensor view of FIG. 31A;

FIG. 32A is an orthographic view illustration of the injection device of FIG. 25, when in an initial operational state, constructed and operative in accordance with a third embodiment of the present invention;

FIG. 32B is a sectional view illustration of the injection device of FIG. 32A;

FIG. 33 is an orthographic view illustration of the injection device of FIG. 25, prior to the needle insertion with the rigid needle shield (RNS) removed, constructed and operative in accordance with a third embodiment of the present invention;

FIG. 34A is an orthographic view illustration of the injection device of FIG. 25 at a later operational state with the plunger rod fully depressed, constructed and operative in accordance with a third embodiment of the present invention;

FIG. 34B is a sectional orthographic view illustration of the injection device of FIG. 34A;

FIG. 35 is a perspective view illustration of an injection device in an assembled configuration, constructed and operative in accordance with a fourth embodiment of the present invention;

FIG. 36 is an exploded perspective view illustration of the injection device of FIG. 35, constructed and operative in accordance with a fourth embodiment of the present invention;

FIG. 37A is a perspective view illustration of the ring element of the injection device of FIG. 35, depicting a proximal surface thereof, constructed and operative in accordance with a fourth embodiment of the present invention;

FIG. 37B is a perspective view illustration of the ring element of FIG. 37A, depicting a proximal surface thereof;

FIG. 38A is a perspective view illustration of the electrical board of the injection device of FIG. 35, depicting a distal surface thereof, constructed and operative in accordance with a fourth embodiment of the present invention;

FIG. 38B is a perspective view illustration of the electrical board of FIG. 38A, depicting a proximal surface thereof;

FIG. 39A is an orthographic view illustration of the rack of the injection device of FIG. 35, constructed and operative in accordance with a fourth embodiment of the present invention;

FIG. 39B is a perspective view illustration of the rack of FIG. 39A;

FIG. 40 is a perspective view illustration of the cap of the injection device of FIG. 35, depicting a proximal view thereof, constructed and operative in accordance with a fourth embodiment of the present invention;

FIG. 41A is a perspective view illustration of the plunger of the injection device of FIG. 35, with the cap removed, constructed and operative in accordance with a fourth embodiment of the present invention;

FIG. 41B is an orthographic view illustration of the plunger view of FIG. 41A;

FIG. 41C is a sectional view illustration of the plunger view of FIG. 41B;

FIG. 42A is an orthographic view illustration of the plunger of the injection device of FIG. 35, in an initial operational state, constructed and operative in accordance with a fourth embodiment of the present invention;

FIG. 42B is a sectional view illustration of the plunger view of FIG. 42A;

FIG. 42C is a detailed view illustration of a first portion of the plunger view of FIG. 42B;

FIG. 42D is a detailed view illustration of a second portion of the plunger view of FIG. 42B;

FIG. 43A is an orthographic view illustration of the plunger of the injection device of FIG. 35, in a subsequent operational state, constructed and operative in accordance with a fourth embodiment of the present invention;

FIG. 43B is a sectional view illustration of the plunger view of FIG. 43A;

FIG. 43C is a detailed view illustration of a first portion of the plunger view of FIG. 43B;

FIG. 43D is a detailed view illustration of a second portion of the plunger view of FIG. 43B;

FIG. 44A is an orthographic view illustration of the plunger of the injection device of FIG. 35, in a yet subsequent operational state, constructed and operative in accordance with a fourth embodiment of the present invention;

FIG. 44B is a sectional view illustration of the plunger view of FIG. 44A;

FIG. 44C is a detailed view illustration of a first portion of the plunger view of FIG. 44B;

FIG. 44D is a detailed view illustration of a second portion of the plunger view of FIG. 44B;

FIG. 45A is an orthographic view illustration of the injection device of FIG. 35 when in an initial operational state, constructed and operative in accordance with a fourth embodiment of the present invention;

FIG. 45B is an orthographic sectional view illustration of the injection device of FIG. 45A;

FIG. 46A is an orthographic view illustration of the injection device of FIG. 35, prior to the needle insertion with the rigid needle shield (RNS) removed, constructed and operative in accordance with a fourth embodiment of the present invention;

FIG. 46B is an orthographic sectional view illustration of the injection device of FIG. 46A;

FIG. 47A is an orthographic view illustration of the injection device of FIG. 35 at a subsequent operational state with the plunger rod partially depressed, constructed and operative in accordance with a fourth embodiment of the present invention;

FIG. 47B is a sectional orthographic view illustration of the injection device of FIG. 47A;

FIG. 48A is an orthographic view illustration of the injection device of FIG. 35 at a later operational state with the plunger rod fully depressed, constructed and operative in accordance with a fourth embodiment of the present invention;

FIG. 48B is a sectional orthographic view illustration of the injection device of FIG. 48A.

### DETAILED DESCRIPTION OF THE EMBODIMENTS

The present invention overcomes the disadvantages of the prior art by providing injection devices with dosage monitoring capabilities. The injection devices of the present invention provide reliable and cost-effective techniques for remotely monitoring the usage of the syringe, including measuring and monitoring dosages of injected substances, which can allow for self-administering of injections, and precluding the need for qualified medical personnel or visitation to a medical facility. The dosage quantities and other injection information can also be monitored and analyzed to facilitate subsequent decisions regarding injections and associated treatments, such as to determine quantities of various medications to manufacture or order, and to monitor proper injections.

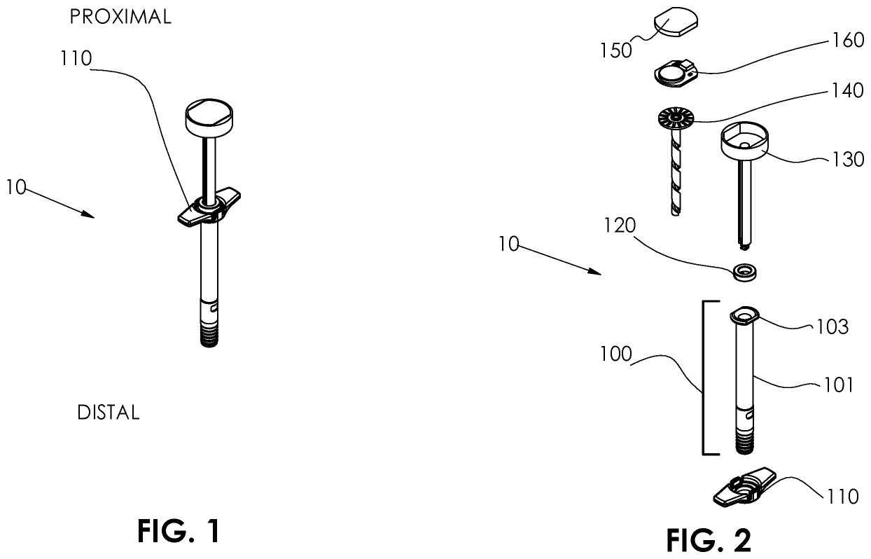

Reference is now made to FIGS. 1 through 12, which collectively illustrate an injection device, generally referenced 10, according to a first embodiment of the present invention. Injection device 10 includes a syringe 100, a gripping unit 110, a ring element 120, a plunger 130, a rotor 140, a cap 150, and an electronics unit 160, shown in an exploded view illustration in in FIG. 2. Injection device 10 has a distal end and a proximal end, which is depicted in the context of FIG. 1, where the distal end faces away from a user holding device 10 and towards the injection site. Injection device 10 is also defined by a longitudinal axis, extending lengthwise along the device between the proximal and distal ends, where an “axial” direction corresponds to a direction parallel to the longitudinal axis (i.e., towards or away from the proximal or distal ends), whereas a “radial” direction corresponds to a direction orthogonal to the longitudinal axis, and extending radially therefrom.

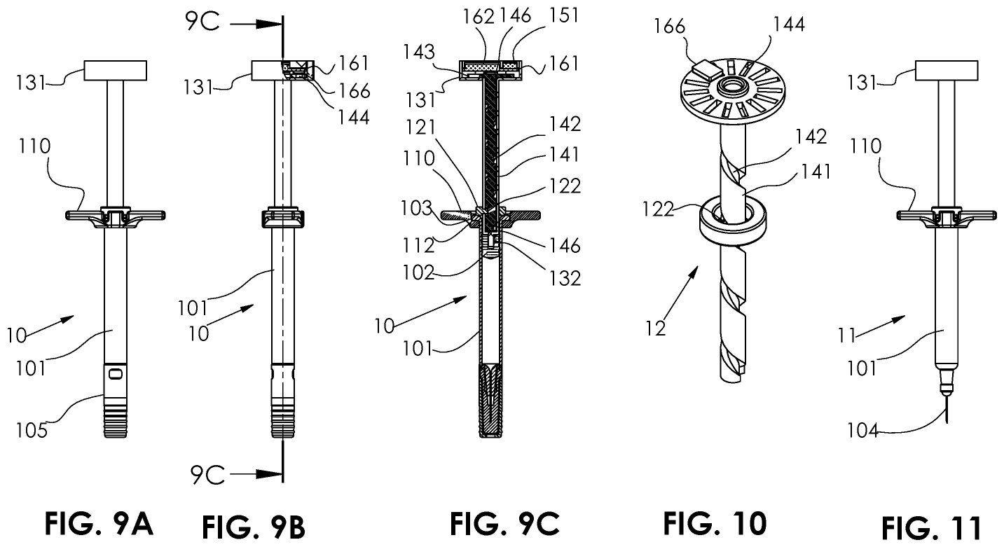

Syringe 100 includes a cylindrical chamber 101, which is bordered at its proximal end by a gripping unit 110. Syringe 100 further includes a needle 104 (shown in FIG. 11) which is positioned within syringe chamber 101 and extends distally from the distal end of syringe 100. Syringe 100 further includes a plunger stopper 102 (shown in FIG. 9C) positioned within syringe chamber 101 and configured to push out the injectant substance (e.g., a drug or medication) through needle 104. Syringe further include a syringe flange 103 (shown in FIG. 9C), which is a short circular protrusion that projects radially outwards at the proximal end of syringe chamber 101. A rigid needle shield (RNS) 105 (FIG. 9A) is configured to cover the distal end of the syringe needle 104 when injection device 100 is not in use, and to be removed before an injection, to maintain sterilization of the needle 104 and the injectant substance and protect against contamination and needlestick injury.

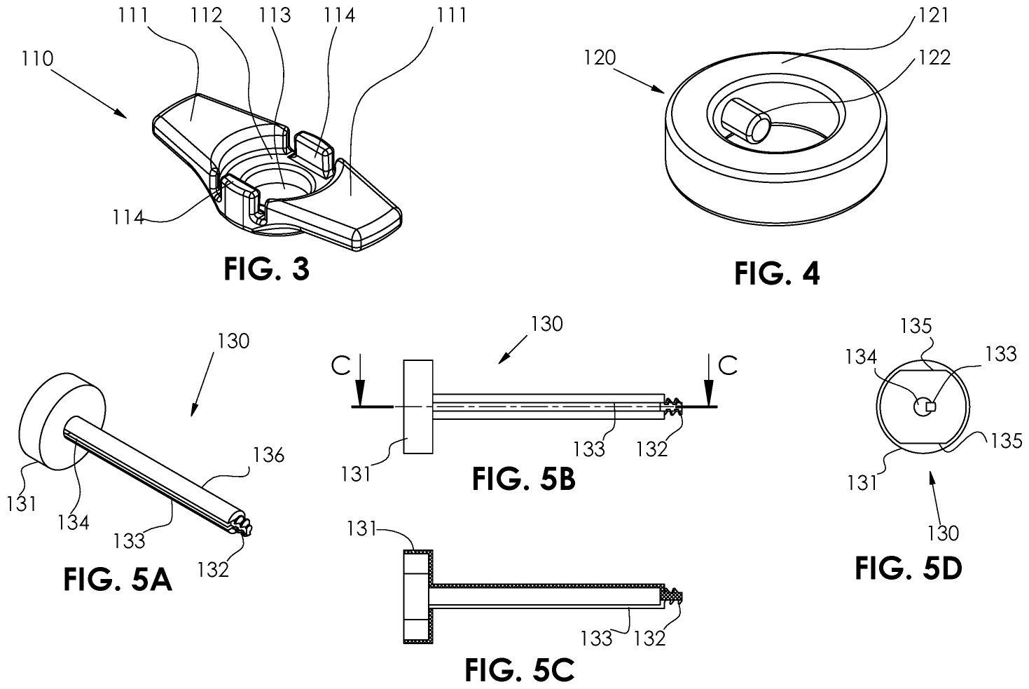

Reference is now made to FIG. 3, which an illustration of gripping unit 110 of injection device 10. Gripping unit 110 includes a pair of finger grippers 111, an annular surface 112, a central opening 113, and a pair of flange snaps 114. Finger grippers 111 are arranged symmetrically on opposing sides. Each finger gripper 111 is a ledge of protrusion that projects radially outwards from gripping unit 110, to enable a user to press his/her fingers against the finger grippers 111 to provide a counterforce when depressing plunger 130. Central opening 113 allows components of injection device 10, such as plunger rod 136, and rotor rod 141, to extend through opening 113 of gripping unit 110 and into the syringe chamber 101, such as during an injection. Snaps 114 are symmetrically arranged on opposing side of surface 112, perpendicularly to finger grippers 111, where each snap 114 is a short protrusion that projects proximally. Snaps 114 are configured to clip onto syringe flange 103 during the assembly process to hold syringe 100 in place relative to gripping unit 110, where syringe flange 103 contacts gripping unit 110 at annular surface 112.

Reference is now made to FIG. 4, which an illustration of ring element 120 of injection device 10. Ring element 120 is annular shaped with a central axial opening and a proximal annular surface 121. A guiding pin 122 extends radially inward from an inner wall of ring element 120. Guiding pin 122 is a fixed solid pin, and is configured to enable the rotation of rotor 140, as will be elaborated upon further hereinbelow. Ring element 120 is disposed at the proximal end of syringe 100. It is noted that ring element 120 may be substantially circular or may be a non-circular annular shape.

FIGS. 5A, 5B, 5C and 5D provide illustrative views of plunger 130 of injection device 10. Plunger 130 is characterized by a tubular rod 136 with a hollow core and aligned longitudinally. Plunger rod 136 is slidably advanceable within the syringe chamber, such that when plunger rod 136 is pushed in a distal direction, such as upon the manual application of force against finger grippers 111 of gripping unit 110, an injectant substance contained within the chamber is ejected distally through the needle aperture. In particular, a plunger rod ending 132 at the distal end of plunger rod 136 is coupled to a syringe piston 102 of syringe 100, such that the distal displacement of plunger rod 136 within syringe chamber 101 causes rod ending 132 to press against syringe piston 102 and forcing the distal displacement of the injectant substance. Plunger 130 includes a plunger head 131 at the proximal end of plunger rod 136. Plunger head 131 is configured as a receptacle, with a circular disc shaped exterior perimeter and an interior perimeter defined by opposing semicircular edges and opposing straight edges (as seen in FIG. 2), and a flat surface orthogonal to the longitudinal axis. Plunger head 131 is configured to contain rotor head 143 and electronics unit 160, and be covered by cap 150. Accordingly, the interior of plunger head 131 is shaped to conform to the perimeter shape of electronics unit 160, such as including straight edges 135 to align with straight edges 153 of cap 150 and straight edges 167 of electronics unit 160 while allowing for rotation of circular rotor head 143. Plunger 130 further includes a longitudinal slot 133 extending axially on an outer surface of plunger rod 136. Guiding pin 122 embeds within longitudinal slot 133 so as to prevent rotation of ring element 120 with respect to the plunger 130, while allowing linear displacement of plunger rod 136. Plunger 130 is characterized with a central aperture 134 extending axially through plunger rod 136. Plunger 130 is concentrically disposed within syringe 100, with ring element 120 encircling plunger rod 136.

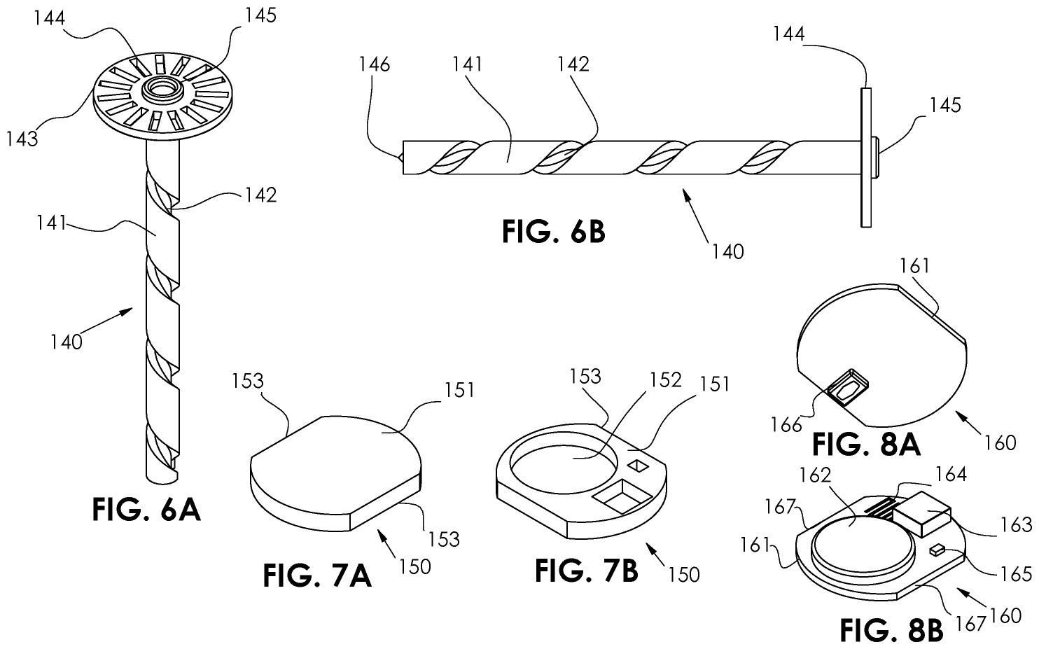

Reference is now made to FIGS. 6A and 6B, which provide illustrative views of rotor 140 of injection device 10. Rotor 140 is characterized by a tubular rod 141 aligned longitudinally and having a screw thread 142 extending in a helical pattern along the length of rod 141. Rotor 140 further includes a rotor head 143 positioned at the proximal end of rod 141, and shaped as a circular disc aligned radially, such that the disc surface is orthogonal to the axial body of rotor rod 141. Rotor head 143 is configured to rotate radially (i.e., about the longitudinal axis). A plurality of rotor head apertures 144 are arranged in a radial pattern along the surface of rotor head 143, such as in the form of multiple rectangular (or other shaped) spacings extending from the core to the perimeter of rotor head 143 and positioned relatively close to one another. Rotor 140 further includes a rotor opening 145, extending axially from the center of rotor head 143 through the core of rotor rod 141, and terminating at a rotor rod ending protrusion 146 positioned at the distal end of rod 141. Rotor rod ending protrusion 146 serves to reduce friction when rotor 140 is rotating within plunger 130 by reducing the surfaces in contact between rotor rod 141 and plunger rod 136. Rotor 140 is concentrically disposed within plunger 130, such that rotor rod 141 is disposed within plunger rod 136 and rotor head 143 is contained within plunger head 131. Guiding pin 122 of ring element 120 interfaces with helical screw thread 142 of rotor rod 141 through plunger rod slot 133, which provides a counterforce to turn rotor rod 141 and cause rotation of rotor 140 when plunger rod 136 is linearly displaced within syringe 100. Ring element 120 is prevented from rotating with respect to plunger 130 by guiding pin 122 being embedded within plunger rod slot 133, which limits guiding pin 122 to linear (axial) motion and prevents rotation of ring element 120.

Reference is now made to FIGS. 7A and 7B, which provide illustrative views of cap 150 of injection device 10. Cap 150 is configured to contain electronics unit 160 and be embedded within plunger head 131. Accordingly, the perimeter of cap 150 is shaped to conform to the perimeter shape of electronics unit 160 and plunger head 131, with a perimeter defined by opposing semicircular edges and opposing straight edges 153 to align with straight edges 167 of electronics unit 160 and straight edges 135 of plunger head 131. Cap 150 includes a flat proximal surface 151 (shown in FIG. 7A). Cap 150 further includes a battery enclosure 152, which is shaped and sized to accommodate a battery of electronics unit 160 (e.g., a circular battery). Cap 150 may further include additional enclosures in the cap interior, which are shaped and sized to accommodate additional components of electronics unit 160 (described further hereinbelow). Cap 150 covers the proximal end of electronics unit 160 and rotor head 143, and is embedded within plunger head 131.

Reference is now made to FIGS. 8A and 8B, which provide illustrative views of electronics unit 160 of injection device 10. Electronics unit 160 includes a main circuit board 161, on which is mounted a battery 162, a controller 163, an antenna 164, an indicator 165, and a sensor 166. Circuit board 161 may also include additional electronic components or sensors not shown, such as: a temperature sensor, a gyroscope, an accelerometer, and an optical sensor. Battery 162 is configured to power at least some of the electronic components on circuit board 161, such as controller 163, indicator 165, and sensor 166. Controller 163 is configured to control the operation of at least some of the electronic components on circuit board, such as to control signal transmission through antenna 164, and to control operation of indicator 165, and sensor 166. Controller 163 may also function as a connectivity module as well, or there may be separate modules. Antenna 164 is configured to transmit and/or receive data signals, such as to transmit a signal representative of injectant dosage information, to a remote location. For example, data signals may be transmitted to a personal computing device (e.g., a smartphone or tablet computer) of a patient or a medical operator, and/or to a data storage unit (e.g., a cloud storage service). Antenna 164 may transmit/receive signals over any suitable data communication channel or network, using any type of channel or network model or any data transmission protocol (e.g., wired, wireless, radio, WiFi, Bluetooth, and the like). Indicator 165 is configured to provide an indication relating to the operation or status of device. For example, indicator 165 may be a visual indicator configured to provide a visual indication, such as an LED, which lights up in a selected manner to reflect a particular device status. For example, indicator 165 may flash or blink at a certain frequency and/or illuminate in a certain color when an injection is in progress, and indicator 165 may flash or blink at a different frequency and/or illuminate in a different color when the injection is completed. Sensor 166 is positioned on circuit board 161 such that the sensor detection surface 166a faces the proximal side of rotor head 143. Sensor 166 is configured to detect at least one property relating to the position and movement of rotor head 143. For example, sensor 166 may be configured to detect a rotational motion of rotor head 143, by detecting the sequential rotation of rotor disc apertures 144 over a selected duration of time. For example, sensor 166 may be configured to detect each time a rotor head aperture 144 passes across the sensor detection surface 166a during the rotation of rotor head 143, by detecting the sequential passage of light through rotor head apertures 144. Alternatively, sensor 166 may be configured to detect a different property relating to the movement of rotor head 143, such as a magnetic change, a capacitance change, an inductance change, an impedance change, an acoustic change, a mechanical detection of rotor head apertures 143, and the like. In general, sensor 166 may be operative to detect and sample electromagnetic radiation at any range of wavelengths (e.g., visible or non-visible light, infrared, ultraviolet, radar, microwave, RF), which may be converted into an electronic signal for subsequent processing and/or transmission. For example, sensor 166 may be embodied by an optical sensor, such as an infrared (IR) sensor. Controller 163 may process the detection samples from sensor 166 and translate the samples into a value representing a linear axial displacement amount of plunger rod 136, which in turn, may be indicative of an ejected dosage of an injected substance contained within syringe 100. Electronics unit 160 is disposed on the proximal end of rotor head 143, and is contained within cap 150 (on the proximal end thereof) and within plunger head 131 (on the distal end thereof). Accordingly, the perimeter of electronics unit 160 is shaped to conform to the perimeter shape of cap 150 and plunger head 131, with a perimeter defined by opposing semicircular edges and opposing straight edges 167 to align with straight edges 153 of cap 150 and straight edges 135 of plunger head 131.

It is noted that the functionality associated with each of the elements of injection device 10 may be distributed among multiple elements or may be performed by other elements of device 10. For example, the functionality described with regard to sensor 166 or controller 163 may be alternatively or additionally implemented by multiple sensor elements or controller elements.

Injection device 10 may optionally include additional components not shown in the Figures. For example, the injection device may include a housing, configured to encase the device components and to allow the user to comfortably hold or grip the injection device. The injection device may further include transparent windows, configured to provide a view of the injectant substance during the injection process.

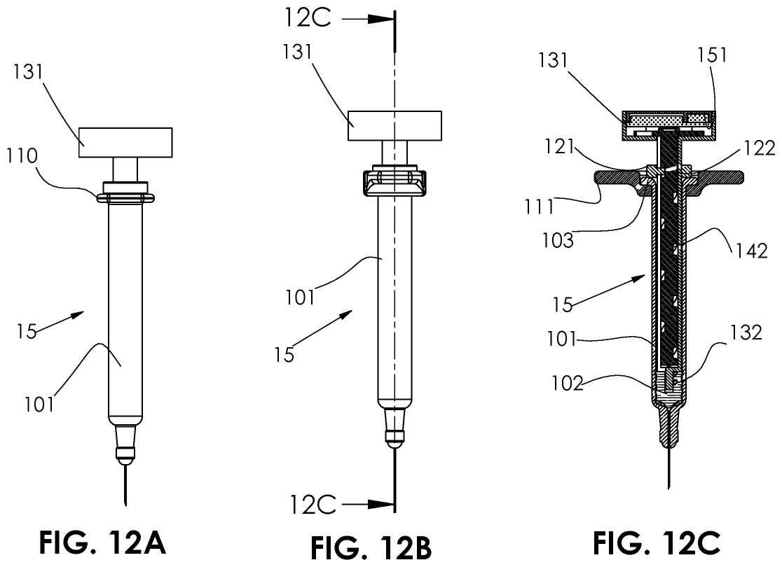

FIGS. 9A, 9B, and 9C provide illustrative views of injection device 10 in an initial operational state, and FIG. 10 illustrates rotor 140 and ring element 120 in the initial operational state. When injection device 10 is in a storage or non-operational state, syringe needle 104 and the distal end of syringe 100 is encased by RNS 105, which acts as a barrier for the injectant substance contained in syringe chamber 101 and keeps the needle and the injectant substance sterile. To perform an injection, a user holds injection device 10, such as with fingers positioned distally against finger grippers 111 of gripping unit 110 and a thumb positioned on a proximal end of plunger head 131. RNS 105 is removed to expose needle 104 (as shown in FIG. 11). The user inserts the exposed distal end of needle 104 into an injection site, such as a body area of a patient to be injected. The user then depresses plunger 130, such as by pressing distally against plunger head 131 while applying a clamping force against finger grippers 111, causing plunger rod 136 to advance in the distal direction within syringe chamber 101. FIGS. 12A, 12B, and 12C provide illustrative views of injection device 10 at a later operational state with the plunger rod fully depressed. The distal advancement of plunger rod 136 propels the injectant substance (shown in FIGS. 9C and 12C) distally within syringe chamber 101 to exit through the distal aperture of needle 104 and enter the injection site.

As plunger rod 136 advances distally within syringe chamber 101, rotor rod 141 (shown in FIG. 10) contained within plunger 130 advances distally as well, resulting in a corresponding rotational movement of rotor 140. In particular, guiding pin 122 engages rotor rod screw thread 142 through plunger rod slot 133, providing a counterforce to turn rotor rod 141 when plunger rod 136 is depressed. Guiding pin 122 is restricted to linear axial motion by plunger rod slot 133 which prevents rotation of ring element 120 with respect to plunger rod 136. The depression of plunger rod 136 distally causes ring element 120 to rest against syringe flange 103 (seen in FIG. 9C), which forces the rotation of rotor 140 due to the aforementioned interactions among rotor rod 141, plunger rod 136, and guiding pin 122. As rotor 140 rotates, sensor 166 detects the amount of rotational movement of rotor head 143, such as by detecting the sequential passage of rotor disc apertures 144 across sensor detection surface 166a. Controller 163 receives and processes the detection samples from sensor 166 to generate a value representative of a linear axial displacement of plunger rod 136. The linear displacement value may then be converted to a value representative of an amount, such as a volume, of injected substance. Injection information, including an injected volume amount, along with other relevant information relating to the injection (e.g., time of injection, drug identification, expiration date, temperature), can then be transmitted via antenna 164 to a remote location, such as to a computing device (e.g., a smartphone or tablet computer) and/or a data storage unit (e.g., a cloud storage service). Alternatively, antenna 164 may transmit the detection samples directly from sensor 166 to a remote server or processor (e.g., at a cloud server), which performs the translation of the samples into linear displacement values and to an injected volume amount. The final injection information may be analyzed by relevant parties to draw conclusions. For example, the injection information may be used by drug manufactures to determine manufacturing quantities for various medications or drugs, or by hospitals or medical clinic staff to determine ordering quantities for such medications, and/or by doctors or medical professional to remotely monitor patient treatment, such as to ensure injections are properly carried out (e.g., to verify that the correct dosage amount was in fact injected, at the correct times and frequencies).

In accordance with an aspect of the present invention, at least some of the elements of injection device 10 may form a unified assembly which may be integrated with an existing syringe (e.g., a separate “add-on device”). For example, ring element 120, plunger 130, rotor 140, cap 150, and/or electronics unit 160, may be configured as a unified device or assembly, which can be integrated with a standard syringe known in the art, such as by replacing a standard plunger and other associated syringe components.

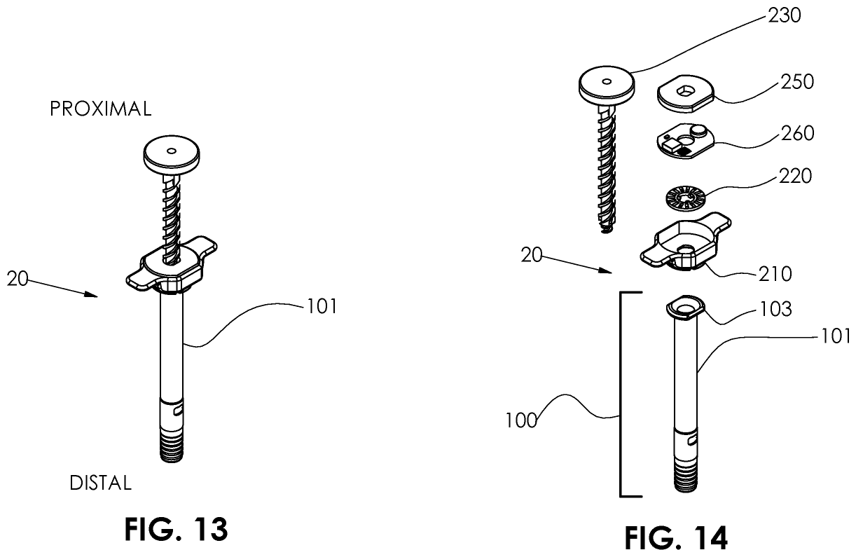

Reference is now made to FIGS. 13 through 24, which collectively illustrate an injection device, generally referenced 20, according to a second embodiment of the present invention. Injection device 20 is generally analogous to injection device 10 of the first embodiment, with selected differences, such as that the electronic components are disposed within the gripping unit in injection device 20, rather than within a plunger head of the plunger as with injection device 10. Furthermore, the guiding pin is embedded with the rotor disc, rather than with a separate ring element, and rather than included a threaded rotor rod, the plunger of injection device 20 is configured with a threaded surface.

Injection device 20 includes a syringe 100, a gripping unit 210, a plunger 230, a rotor 220, a cap 250, and an electronics unit 260, shown in an exploded view illustration in in FIG. 14. Injection device 20 has a distal end and a proximal end, which is depicted in FIG. 13, where the distal end faces away from a user holding device 20 and towards the injection site. Injection device 20 is also defined by a longitudinal axis, extending lengthwise along the assembly between the proximal and distal ends, where an “axial” direction corresponds to a direction parallel to the longitudinal axis (i.e., towards or away from the proximal or distal ends), whereas a “radial” direction corresponds to a direction orthogonal to the longitudinal axis, and extending radially therefrom.

Syringe 100 of injection device 20 is a standard syringe which is identical to syringe 100 of injection device 10.

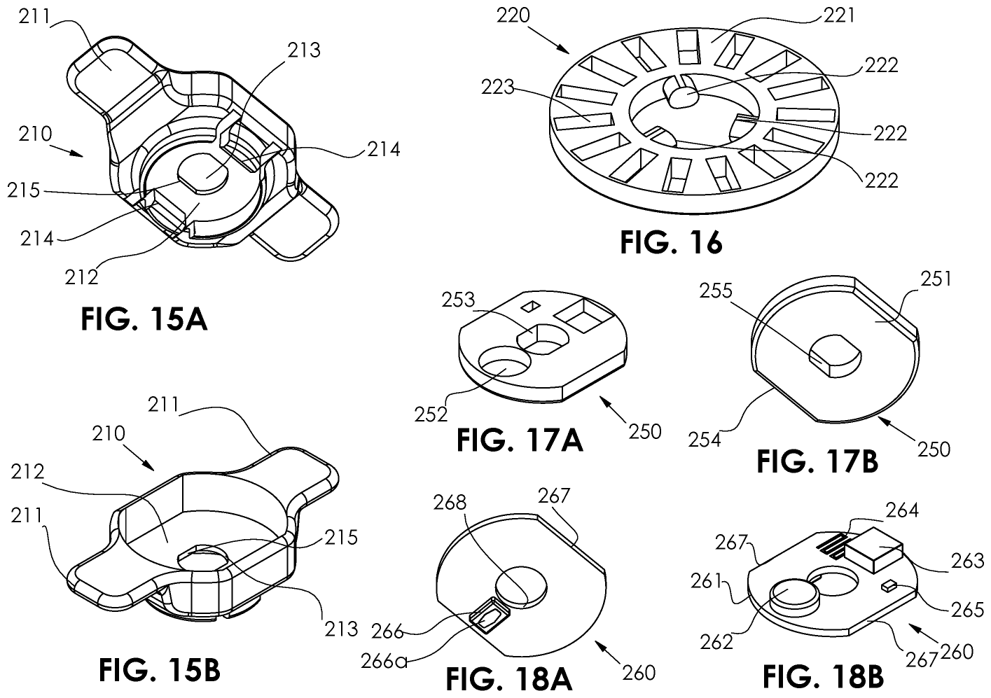

Gripping unit 210 of injection device 20 (illustrated in FIGS. 15A and 15B) is similar to gripping unit 100 of injection device 10 (illustrated in FIG. 3). Gripping unit 210 includes a pair of finger grippers 211, an annular surface 212, a central opening 213, and a pair of snaps 214. Finger grippers 211 are arranged symmetrically on opposing sides. Each finger gripper 211 is a ledge or protrusion that projects radially outwards from gripping unit 210, to enable a user to press his/her fingers against the finger grippers 211 to provide a counterforce when depressing plunger 230. Central opening 213 allows components of injection device 20, such as plunger rod 231, to extend through gripping unit 210 into the syringe chamber 101, such as during an injection. Accordingly, central opening 213 is sized and shaped to conform to the cross-sectional size and shape of plunger rod 231, so as to allow plunger rod 231 to pass therethrough. For example, central opening 213 includes opposing straight edges 215, to align with straight edges 235 of plunger 230. Snaps 114 are symmetrically arranged on opposing sides of surface 212, perpendicularly to finger grippers 211, where each snap 214 is a short protrusion that projects proximally. Snaps 214 are configured to clip onto syringe flange 103 during the assembly process to hold syringe 100 in place relative to gripping unit 210, where syringe flange 103 contacts gripping unit 210 at annular surface 212.

Reference is now made to FIG. 16, which an illustration of rotor 220 of injection device 20. Rotor 220 includes a circular disc shaped rotor surface 221 aligned orthogonal to the axial direction. A plurality of rotor apertures 223 are arranged in a radial pattern along rotor surface 221, such as in the form of multiple rectangular (or other shaped) spacings extending from the core to the perimeter of rotor surface 221 and positioned relatively close to one another. Rotor 220 is characterized by a central opening, with a diameter at least large enough to accommodate the diameter of plunger rod 231. Rotor 220 further includes one or more guiding pins 222, extending radially inward from an inner wall of rotor 240. For example, rotor 220 may include three guiding pins 222 equidistant from one another. Rotor 220 is configured to rotate radially (i.e., about the longitudinal axis). Rotor 220 is contained within gripping unit 210, disposed on the proximal end of gripping unit surface 212, and positioned distally from electronics unit 260 and cap 250.

Reference is now made to FIGS. 17A and 17B, which provide illustrative views of cap 250 of injection device 20. Cap 250 is configured to contain electronics unit 260 and be embedded within gripping unit 210. Accordingly, the perimeter of cap 250 is shaped to conform to the perimeter shape of electronics unit 260 and gripping unit 210, with a perimeter defined by opposing semicircular edges and opposing straight edges 254 to align with straight edges 267 of electronics unit 260 and straight edges of gripping unit 210. Cap 250 includes a flat proximal surface 251 (shown in FIG. 17B). Cap 250 further includes a battery enclosure 252, which is shaped and sized to accommodate a battery of electronics unit 260 (e.g., a circular battery). Cap 250 further includes a central opening 253, which is shaped and sized to accommodate plunger rod 231, such as including straight edges 255 to align with straight edge 235 of plunger rod 231. Accordingly, battery enclosure 252 may be smaller than battery enclosure 152 of cap 150 of device 10, so as to accommodate central opening 253 (which is not present in cap 150). Cap 250 may further include additional enclosures in the cap interior, which are shaped and sized to accommodate additional components of electronics unit 260 (described further hereinbelow). Cap 250 covers the proximal end of electronics unit 260 and rotor 220, and is embedded within gripping unit 210.

Reference is now made to FIGS. 18A and 18B, which provide illustrative views of electronics unit 260 of injection device 20. Electronics unit 260 includes a main circuit board 261, on which is mounted a battery 262, a controller 263, an antenna 264, an indicator 265, and a sensor 266. Electronics unit 260 is generally analogous to electronics unit 160 of injection device 10 (illustrated in FIGS. 8A and 8B), but electronics unit 260 further includes a central opening 268, which is shaped and sized to accommodate plunger rod 231. Accordingly, battery 262 may be smaller than battery 162, so as to accommodate central opening 268 (which is not present in electronics unit 160). Sensor 266 is configured to detect at least one property relating to the position and movement of rotor 220. For example, sensor 266 may be configured to detect a rotational motion of rotor 220, such as by detecting the sequential rotation of rotor apertures 223 over time, such as by detecting the sequential passage of light onto the sensor detection surface 266a through a selected rotor aperture 223. Alternatively, sensor 266 may be configured to detect a different property relating to the movement of rotor 220, such as a magnetic change, a capacitance change, an inductance change, an impedance change, an acoustic change, a mechanical detection of rotor apertures 223, and the like. In general, sensor 266 may be operative to detect and sample electromagnetic radiation at any range of wavelengths (e.g., visible or non-visible light, infrared, ultraviolet, radar, microwave, RF), which may be converted into an electronic signal for subsequent processing and/or transmission. Electronics unit 260 is disposed on the proximal end of rotor 220, and is contained within cap 250 (on the distal end thereof) and within gripping unit 210 (on the proximal end thereof). Accordingly, the perimeter of electronics unit 260 is shaped to conform to the perimeter shape of cap 250 and gripping unit 210, with a perimeter defined by opposing semicircular edges and opposing straight edges 267 to align with straight edges of cap 250 and gripping unit 210, respectively.

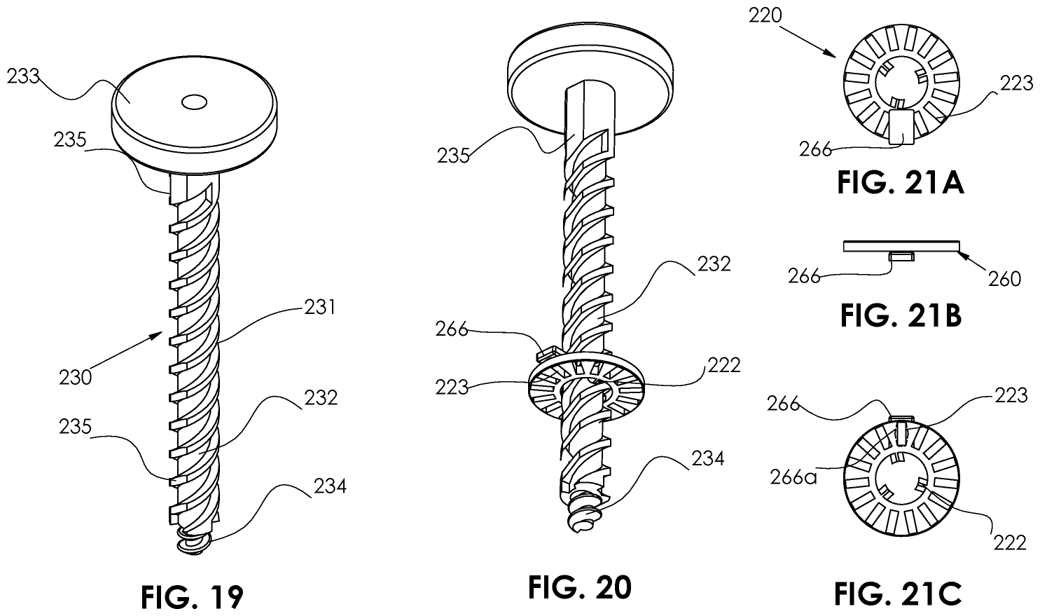

Reference is now made to FIGS. 19 and 20, which provide illustrative views of plunger 230 of injection device 20. Plunger 230 includes a tubular plunger rod 231 aligned longitudinally. Plunger rod 231 is characterized by a screw thread 232 extending in a helical pattern along the length of rod 231. A plunger finger press 233 is positioned at the proximal end of plunger 230 and characterized by a circular disc defining a radial surface orthogonal to the longitudinal axis. Plunger rod 231 is slidably advanceable within syringe chamber 101, such that when plunger 230 is pushed in a distal direction, such as upon the manual application of force against plunger finger press 231 and finger grippers 211, an injectant substance contained within syringe chamber 101 is ejected distally through the needle aperture. In particular, a plunger rod ending 234, at the distal end of plunger rod 231, is coupled to a syringe piston 102 of syringe 100, such that the distal displacement of plunger rod 231 within syringe chamber 100 causes plunger rod ending 234 to press against syringe piston 102 and forcing a distal displacement of the injectant substance. Plunger 230 is concentrically disposed within syringe 100, such that plunger rod 231 extends axially through central opening 213 of gripping unit 210, through the central opening of rotor 220, through central opening 253 of cap 250, and through central opening 268 of electronics unit 260. Straight edge 235 of plunger rod 231 is a straight flat edge surface aligned with straight edge 215 of gripping unit 210 and straight edge 255 of cap 250, so as to prevent rotation of plunger rod 231 relative to gripping unit 210, and prevent a corresponding rotation of rotor 220, which may result in false detection readings by sensor 266.

FIGS. 21A, 21B, and 21C provide illustrative views of rotor 220 and sensor 266 of injection device 20.

It is noted that the functionality associated with each of the elements of injection device 20 may be distributed among multiple elements or may be performed by other elements of device 20. Injection device 20 may optionally include additional components not shown in the Figures. For example, the injection device may include a housing, configured to encase the device components and to allow the user to comfortably hold or grip the injection device. The injection device may further include transparent windows, configured to provide a view of the injectant substance during the injection process.

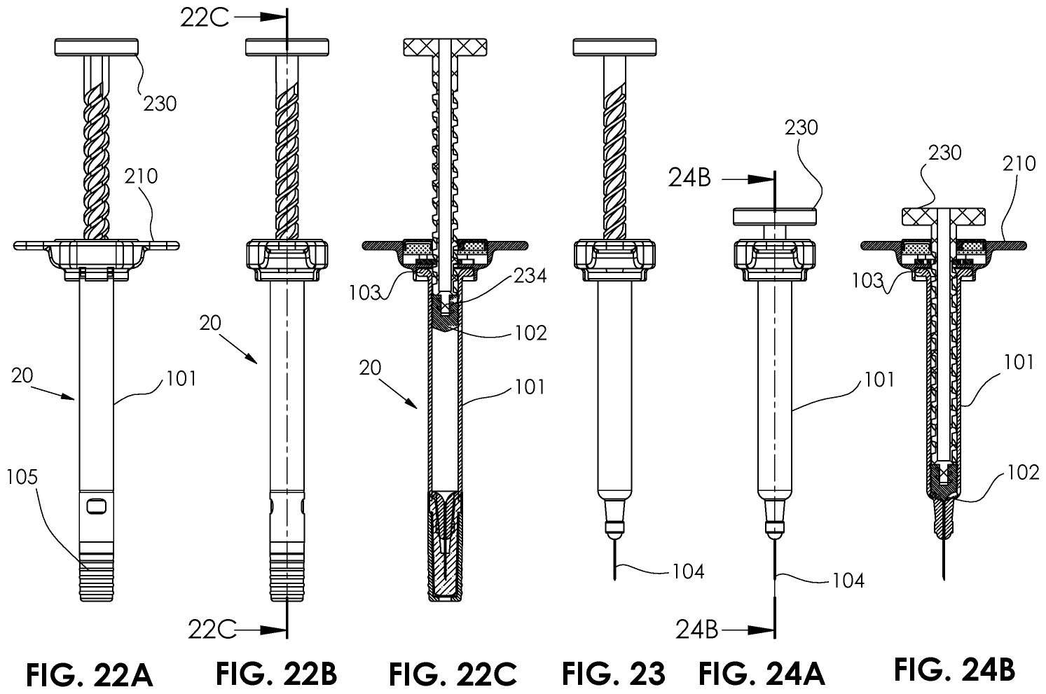

FIGS. 22A, 22B, and 22C provide illustrative views of injection device 20 in an initial operational state. When injection device 20 is in a storage or non-operational state, syringe needle 104 and the distal end of syringe 100 is encased by RNS 105. To perform an injection, a user holds injection device 20, such as with fingers positioned distally against finger grippers 211 of gripping unit 210 and a thumb positioned against plunger finger press 233. RNS 105 is removed to expose needle 104 (as shown in FIG. 23). The user inserts the exposed distal end of needle 104 into an injection site, such as a body area of a patient to be injected. The user then depresses plunger 230, such as by pressing distally against plunger finger press 233 while applying a clamping force against finger grippers 211, causing plunger rod 231 to advance in a distal direction within syringe chamber 101. FIGS. 24A and 24B provide illustrative views of injection device 20 at a later operational state with the plunger rod fully depressed. The distal advancement of plunger rod 231 propels the injectant substance distally within syringe chamber 101 to exit through the distal aperture of needle 104 and enter the injection site.

The linear distal displacement of plunger rod 231 within syringe chamber 101 results in a corresponding rotational movement of rotor 220. In particular, guiding pins 222 of rotor 220 engages plunger rod screw thread 232, providing a counterforce to turn rotor 220 relative to plunger rod 231. As rotor 220 rotates, sensor 266 detects the amount of rotational movement of rotor 220, such as by detecting the sequential passage of rotor disc apertures 223 across sensor detection surface 266a. Plunger rod 231 is restricted from rotating with respect to gripping unit 210 due to the orientation of plunger rod straight edge 235 and shape of gripping unit central opening 213, which prevents sensor 266 from detecting irrelevant rotations of rotor 220 during a non-injection. Controller 263 receives and processes the detection samples from sensor 266 to generate a value representative of a linear axial displacement of plunger rod 231. The linear displacement value may then be converted to a value representative of an amount, such as a volume, of injected substance. Injection information, including an injected volume amount, along with other relevant information relating to the injection (e.g., time of injection, drug identification, expiration date, temperature), can then be transmitted via antenna 264 to a remote location, such as to a computing device (e.g., a smartphone or tablet computer) and/or a data storage unit (e.g., a cloud storage service). Alternatively, antenna 264 may transmit the detection samples directly from sensor 266 to a remote server or processor (e.g., at a cloud server), which performs the translation of the samples into linear displacement values and to an injected volume amount. The final injection information may be analyzed by relevant parties to draw conclusions. For example, the injection information may be used by drug manufactures to determine manufacturing quantities for various medications or drugs, or by hospitals or medical clinic staff to determine ordering quantities for such medications, and/or by doctors or medical professionals to remotely monitor patient treatment, such as to ensure injections are properly carried out (e.g., to verify that the correct dosage amount was in fact injected, at the correct times and frequencies). In accordance with an aspect of the present invention, at least some of the elements of injection device 20 may form a unified assembly which may be integrated with an existing syringe (e.g., a separate “add-on device”). For example, gripping unit 210, plunger 230, rotor 220, cap 250, and/or electronics unit 260, may be configured as a unified device or assembly, which can be integrated with a standard syringe known in the art, such as by replacing a standard plunger and other associated syringe components.

It will be appreciated that injection devices 10 and 20 allows for remote processing and analysis of information relating to an injection process, such as quantities, times and frequencies of injection dosages. The injection information can also be monitored and analyzed to facilitate subsequent decisions regarding injections and associated treatments, such as to determine quantities of various medications to manufacture or order, and to enable remote monitoring of injections by doctors or health care personnel. For example, injection devices 10 and 20 may provide remote monitoring and verification of the injection process, by allowing verification that an injection was properly carried out, with the correct dosage amount and at a correct time and frequency.

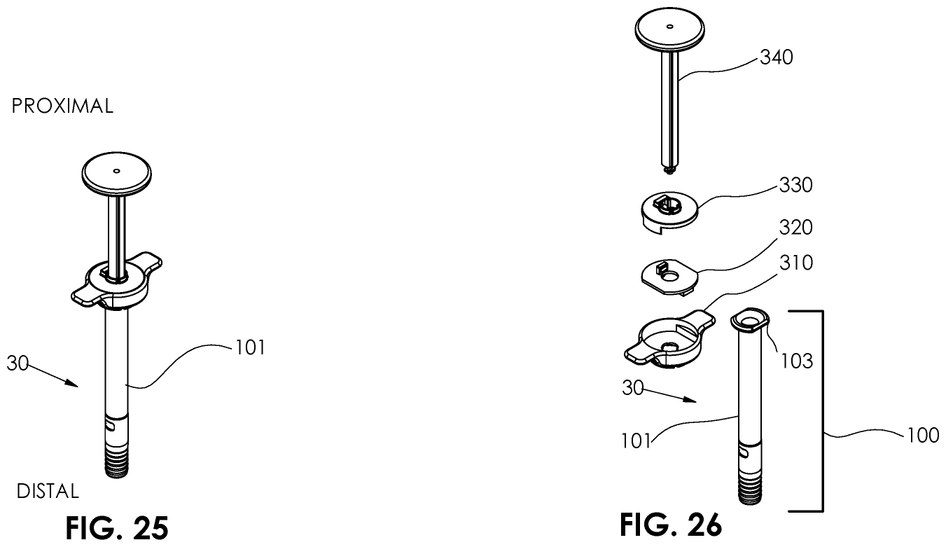

Reference is now made to FIGS. 25 through 34, which collectively illustrate an injection device, generally referenced 30, according to a third embodiment of the present invention. Injection device 30 includes a syringe 100, a gripping unit 310, an electronics unit 320, a cap 330, and a plunger 340, shown in an exploded view illustration in in FIG. 26. Injection device 30 has a distal end and a proximal end, which is depicted in FIG. 25, where the distal end faces away from a user holding device 30 and towards the injection site. Injection device 30 is also defined by a longitudinal axis, extending lengthwise along the assembly between the proximal and distal ends, where an “axial” direction corresponds to a direction parallel to the longitudinal axis (i.e., towards or away from the proximal or distal ends), whereas a “radial” direction corresponds to a direction orthogonal to the longitudinal axis, and extending radially therefrom.

Syringe 100 of injection device 30 is a standard syringe which is identical to syringe 100 of injection device 10.

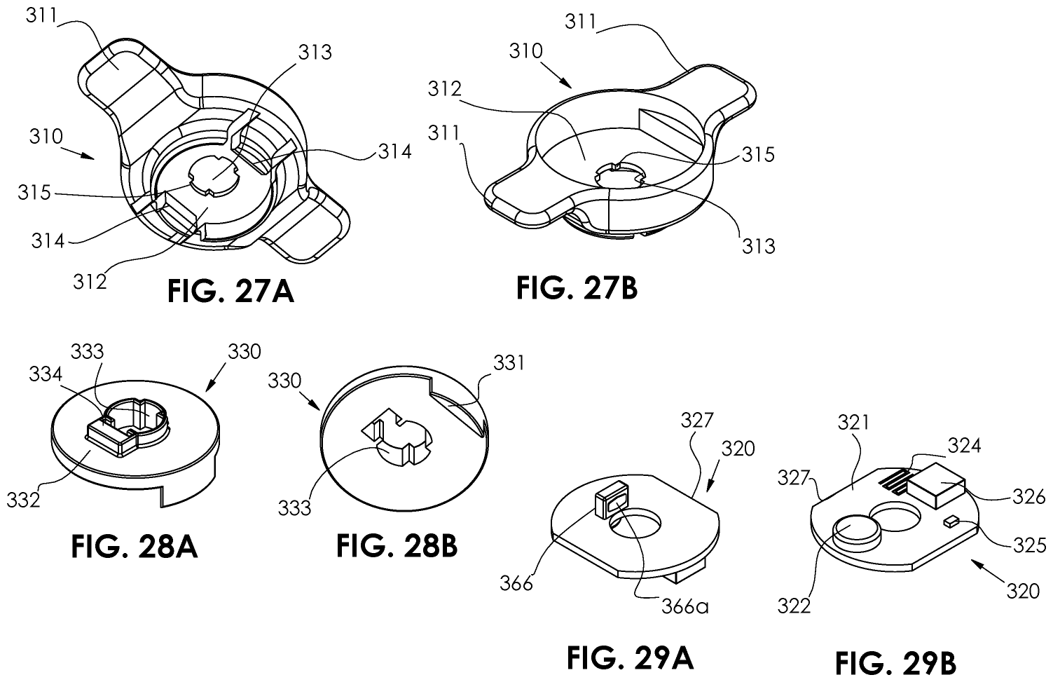

Gripping unit 310 of injection device 30 (illustrated in FIGS. 27A and 27B) is similar to gripping unit 210 of injection device 20 (illustrated in FIGS. 15A and 15B). Gripping unit 310 includes a pair of finger grippers 311, an annular surface 312, a central opening 313, and a pair of snaps 314. Finger grippers 311 are arranged symmetrically on opposing sides, where each finger gripper 311 is a ledge of protrusion that projects radially outwards from gripping unit 310, to enable a user to press his/her fingers against the finger grippers 311 to provide a counterforce when depressing plunger 340. Central opening 313 allows plunger rod 341 to extend through gripping unit 310 into the syringe chamber 101, such as during an injection. Accordingly, central opening 313 is sized and shaped to conform to the cross-sectional size and shape of plunger rod 341, so as to allow plunger rod 341 to pass therethrough. For example, central opening 313 includes opposing edges 315 to align with teeth 342 of plunger rod 341. Snaps 314 are symmetrically arranged on opposing sides of surface 312, perpendicularly to finger grippers 311, where each snap 314 is a short protrusion that projects proximally. Snaps 314 are configured to clip onto syringe flange 103 during the assembly process to hold syringe 100 in place relative to gripping unit 310, where syringe flange 103 contacts gripping unit 310 at annular surface 312.

Reference is now made to FIGS. 28A and 28B, which provide illustrative views of cap 330 of injection device 30. Cap 330 is configured to contain electronics unit 320 and be embedded within gripping unit 310. Accordingly, the perimeter of cap 330 is shaped to conform to the perimeter shape of electronics unit 320 and gripping unit 310. Cap 330 includes a flat proximal surface 332 (shown in FIG. 28A). Cap 330 includes a rib 331 which protrudes distally from cap surface 332 and is configured to slide inside a corresponding slot within gripping unit 310 while extending over an edge 327 of electronics unit 320. Cap 330 further includes a sensor enclosure 334, which is shaped and sized to accommodate sensor 366 of electronics unit 320. Cap 330 may further include additional enclosures in the cap interior, which are shaped and sized to accommodate additional components of electronics unit 320. Cap 330 further includes a central opening 333 shaped and sized to accommodate the cross-sectional shape of plunger rod 341, so as to allow plunger rod 341 to pass through central opening 333. Cap 330 covers the proximal end of electronics unit 320 and is embedded within gripping unit 310.

Reference is now made to FIGS. 29A and 29B, which provide illustrative views of electronics unit 320 of injection device 30. Electronics unit 320 includes a main circuit board 321, on which is mounted a battery 322, an antenna 324, an indicator 325, a controller 326, and a sensor 366. Electronics unit 320 is generally analogous to electronics unit 260 of injection device 20 (illustrated in FIGS. 18A and 18B), but the central opening of electronics unit 320 is sized and shaped to accommodate the plunger rod 341. Sensor 366 is configured to detect at least one property relating to the position and movement of teeth 342 of plunger rod 341. For example, sensor 366 may be configured to detect an axial displacement of plunger rod 341 by detecting the sequential displacement of teeth 342, as will be described further hereinbelow. Sensor 366 may be embodied by an optical sensor device, or by an alternative type of sensor configured to detect a property relating to the movement of teeth 342 of plunger rod 341, such as a magnetic change, a capacitance change, an inductance change, an impedance change, an acoustic change, a mechanical change, and the like. In general, sensor 366 may be operative to detect and sample electromagnetic radiation at any range of wavelengths (e.g., visible or non-visible light, infrared, ultraviolet, radar, microwave, RF), which may be converted into an electronic signal for subsequent processing and/or transmission. Electronics unit 320 is contained within cap 330 (on the distal end thereof), and within gripping unit 310 (on the proximal end thereof).

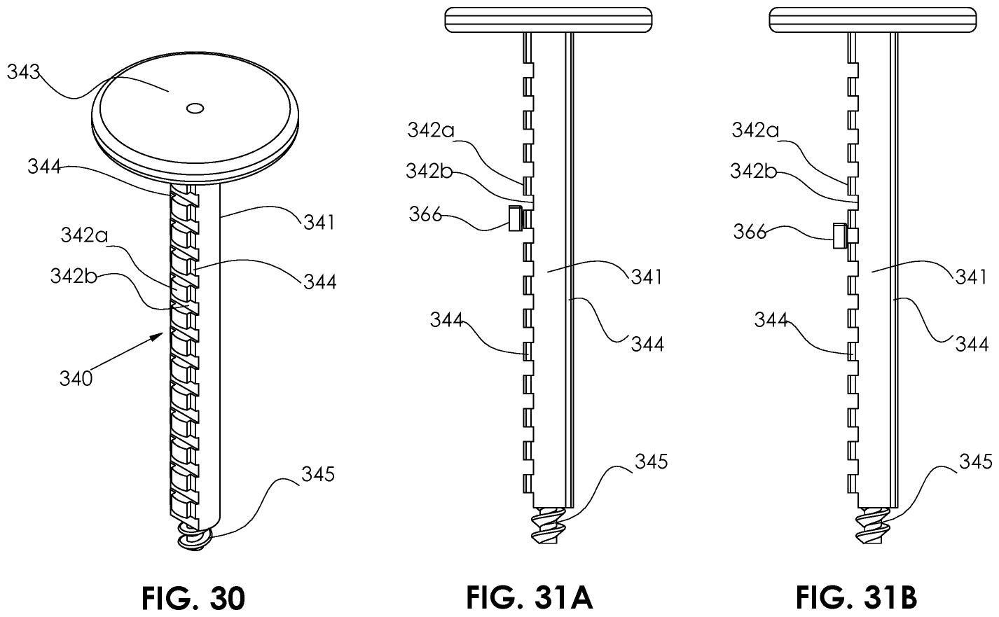

Reference is now made to FIGS. 30, 31A and 31B, which provide illustrative views of plunger 340 and sensor 366 of injection device 30. Plunger 340 includes a tubular plunger rod 341 aligned longitudinally. Plunger rod 341 is characterized by a strip of teeth 342 extending axially along a portion of the length of plunger rod 341, while the remaining portion of plunger rod 341 has a substantially circular radial diameter. Teeth strip 342 is characterized by a series of tooth protrusions 342a interspersed with a series of tooth indentations 342b, such that a respective tooth protrusion 342a is followed by a respective tooth indentation 342b which is followed by another tooth protrusion 342a, and so forth. Each tooth protrusion 342a protrudes radially outwards from teeth strip 342, and each tooth indentation 342b is indented radially inwards. Teeth strip 342 further includes a straight edge surface 344, aligned with straight edges 315 of gripping unit 310, so as to prevent rotation of plunger rod 341 relative to gripping unit 310, and prevent false detection readings by sensor 366. A plunger finger press 343 is positioned at the proximal end of plunger 340. Finger press 343 is characterized by a circular disc defining a radial surface orthogonal to the longitudinal axis of plunger 340. Plunger rod 341 is slidably advanceable within syringe chamber 101, such that when plunger 340 is pushed in a distal direction, such as upon the manual application of force against plunger finger press 343 and finger grippers 311, an injectant substance contained within syringe chamber 101 is ejected distally through the needle aperture. In particular, a plunger rod ending 345, at the distal end of plunger rod 341, is coupled to a syringe piston 102 of syringe 100, such that the distal displacement of plunger rod 341 within syringe chamber 101 causes plunger rod ending 345 to press against syringe piston 102 and forcing a distal displacement of the injectant substance. Plunger 340 is concentrically disposed within syringe 100, such that plunger rod 341 extends axially through central opening 313 of gripping unit 310, through central opening 333 of cap 330, and through the central opening of electronics unit 320.

It is noted that the functionality associated with each of the elements of injection device 30 may be distributed among multiple elements or may be performed by other elements of device 30. Injection device 30 may optionally include additional components not shown in the Figures. For example, the injection device may include a housing, configured to encase the device components and to allow the user to comfortably hold or grip the injection device. The injection device may further include transparent windows, configured to provide a view of the injectant substance during the injection process.

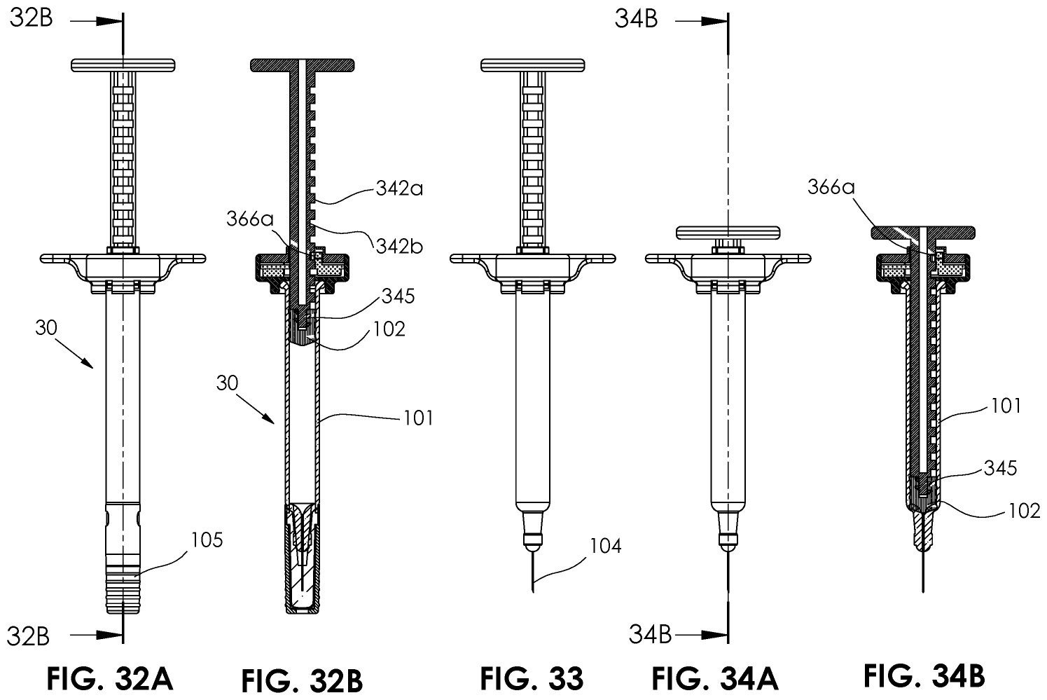

FIGS. 32A, 32B, 33, 34A and 34B provide illustrative views of injection device 30 in an initial operational state. When injection device 30 is in a storage or non-operational state, syringe needle 104 and the distal end of syringe 100 is encased by RNS 105. To perform an injection, a user holds injection device 30, such as with fingers positioned distally against finger grippers 311 of gripping unit 310 and a thumb positioned against plunger finger press 343. RNS 105 is removed to expose needle 104 (as shown in FIG. 33). The user inserts the exposed distal end of needle 104 into an injection site, such as a body area of a patient to be injected. The user then depresses plunger 340, such as by pressing distally against plunger finger press 343 while applying a clamping force against finger grippers 311, causing plunger rod 341 to advance in a distal direction within syringe chamber 101. FIGS. 34A and 34B provide illustrative views of injection device 30 at a later operational state with the plunger rod fully depressed. The distal advancement of plunger rod 341 propels the injectant substance distally within syringe chamber 101 to exit through the distal aperture of needle 104 and enter the injection site.

The linear distal displacement of plunger rod 341 within syringe chamber 101 results in the passage of teeth strip 342 through the openings of cap 330 and electronics unit 320, and across the detection surface 366a of sensor 366, which remains fixed within the stationary gripping unit 310. During the linear advancement of plunger rod 341, sensor 366 detects the displacement of teeth strip 342, such as by detecting the sequential passage of tooth protrusions 342a across sensor detection surface 366a. Plunger rod 341 is restricted from rotating with respect to gripping unit 310 due to the orientation of teeth strip straight edge 344 and the shape of gripping unit central opening 313, which prevents irrelevant detection readings by sensor 366. The sensor readings are received and processed by controller 326 to generate a value representative of a linear axial displacement of plunger rod 341, which in turn may be converted to a value representative of an amount, such as a volume, of injected substance using a suitable conversion formula. Injection information, including an injected volume amount, along with other relevant information relating to the injection (e.g., time of injection, drug identification, expiration date, temperature), can then be transmitted via antenna 324 to a remote location, such as to a computing device (e.g., a smartphone or tablet computer) and/or a data storage unit (e.g., a cloud storage service). Alternatively, antenna 324 may transmit the detection samples directly from sensor 366 to a remote server or processor (e.g., at a cloud server), which performs the translation of the samples into linear displacement values and an injected volume amount. The final injection information may be analyzed by relevant parties to draw conclusions. For example, the injection information may be used by drug manufactures to determine manufacturing quantities for various medications or drugs, or by hospitals or medical clinic staff to determine ordering quantities for such medications, and/or by doctors or medical professionals to remotely monitor patient treatment, such as to ensure injections are properly carried out (e.g., to verify that the correct dosage amount was in fact injected, at the correct times and frequencies).

In accordance with an aspect of the present invention, at least some of the elements of injection device 30 may form a unified assembly which may be integrated with an existing syringe (e.g., a separate “add-on device”). For example, gripping unit 310, electronics unit 320, cap 330 and/or plunger 340, may be configured as a unified device or assembly, which can be integrated with a standard syringe known in the art, such as by replacing a standard plunger and other associated syringe components.

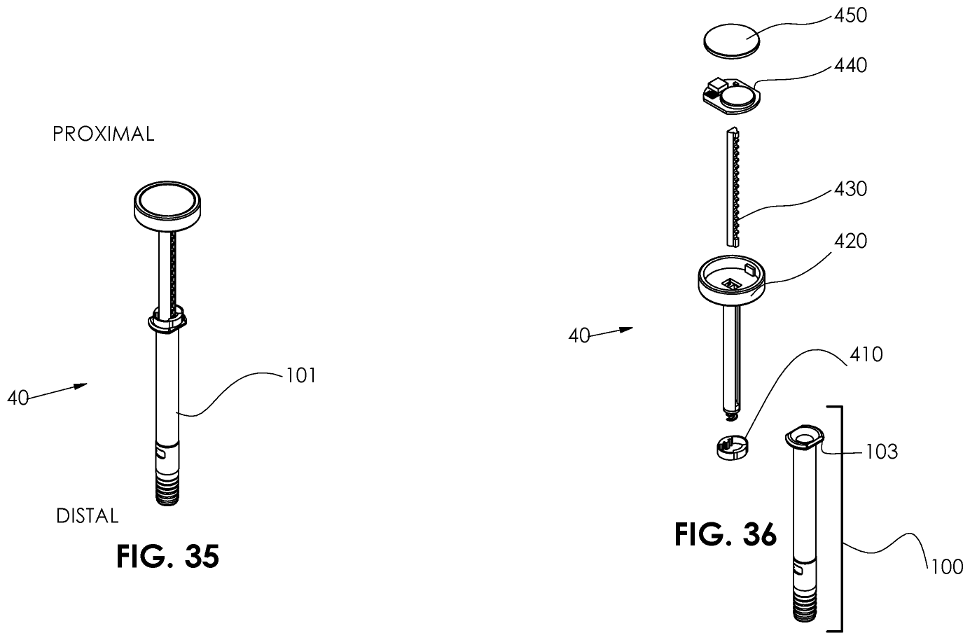

Reference is now made to FIGS. 35 through 48, which collectively illustrate an injection device, generally referenced 40, according to a fourth embodiment of the present invention. Injection device 40 includes a syringe 100, a flexible ring 410, a plunger 420, a rack 430, an electronics unit 440, and a cap 450, shown in an exploded view illustration in FIG. 36. Injection device 40 has a distal end and a proximal end, which is depicted in FIG. 35, where the distal end faces away from a user holding device 40 and towards the injection site. Injection device 40 is also defined by a longitudinal axis, extending lengthwise along the assembly between the proximal and distal ends, where an “axial” direction corresponds to a direction parallel to the longitudinal axis (i.e., towards or away from the proximal or distal ends), whereas a “radial” direction corresponds to a direction orthogonal to the longitudinal axis, and extending radially therefrom.

Syringe 100 of injection device 40 is a standard syringe which is identical to syringe 100 of injection device 30.

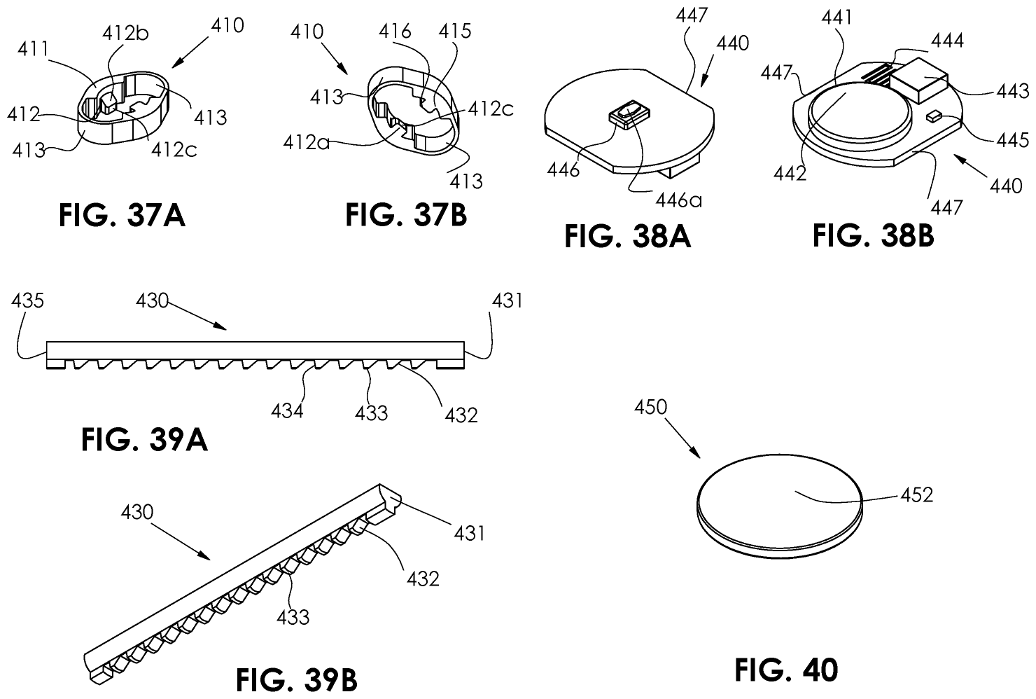

Reference is now made to FIGS. 37A and 37B, which provide illustrative views of flexible ring 410 of injection device 40. Ring 410 includes a proximal ring surface 411 facing a proximal side of ring 410, and a distal ring surface 415 facing a distal side of ring 410. A first ring tooth 412 projects radially inwards from an inner surface of ring 410. Ring tooth 412 includes a flat distal tooth surface 412a facing a distal direction, an inclined proximal tooth surface 412b facing a proximal direction, and an inner tooth surface facing radially inwards. A second ring tooth 416 projects radially inwards from an inner surface of ring 410 opposite first ring tooth 412. Ring 410 further includes flexible portions 413, arranged on opposing sides, with the surfaces of flexible portions 413 facing radially such that flexible portions 413 are configured to bend or deform radially inwards or radially outwards. Ring 410 is disposed around plunger rod 421 of plunger 420, at a proximal end of syringe 100.

Reference is now made to FIGS. 38A and 38B, which provide illustrative views of electronics unit 440 of injection device 40. Electronics unit 440 includes a main circuit board 441, on which is mounted a battery 442, an antenna 444, an indicator 445, a controller 443, and a sensor 446. Electronics unit 420 is generally analogous to electronics unit 160 of injection device 10 (illustrated in FIGS. 8A and 8B). Circuit board 441 may also include additional electronic components not shown. Battery 442 is configured to power at least some of the electronic components on circuit board 441 (such as controller 443, indicator 445, and sensor 446). Controller 443 is configured to control the operation of at least some of the electronic components on circuit board, such as to control antenna 444, indicator 445 and/or sensor 446. Antenna 444 is configured to transmit and/or receive data signals, such as to transmit a signal representative of injectant dosage information, to a remote location. Sensor 466 is configured to detect at least one property relating to the position and movement of rack 430. For example, sensor 446 may be configured to detect an axial displacement of rack 430, by detecting the sequential displacement of rack teeth 433, as will be described further hereinbelow. Sensor 446 may be embodied by an optical sensor device, or by an alternative type of sensor configured to detect a property relating to the movement of rack teeth 433, such as a magnetic change, a capacitance change, an inductance change, an impedance change, an acoustic change, a mechanical change, and the like. Indicator 445 is configured to provide an indication relating to the operation or status of device. For example, indicator 445 may be a visual indicator configured to provide a visual indication, such as an LED, which lights up in a selected manner to reflect a particular device status. Electronics unit 440 is covered by cap 450 (on the distal end thereof), and within plunger head 426 of plunger 420 (on the proximal end thereof).

Reference is now made to FIGS. 39A and 39B, which provide illustrative views of rack 430 of injection device 40. Rack 430 is disposed longitudinally within plunger 420. Rack 430 includes a proximal rack surface 435 facing a proximal side of rack 430, and a distal rack surface 431 facing a distal side of rack 430. Rack 430 further includes a series of teeth 433 extending axially along the length of rack 430, where teeth 430 are embodied by triangular shaped protrusions that project radially outwards when rack is positioned within plunger 420. Each tooth 433 includes an inclined proximal tooth face 432 facing a proximal direction, and a distal tooth face 434 facing a distal direction.

Cap 450 of injection device 40 (illustrated in FIG. 40) is configured to cover electronics unit 440 and be embedded within plunger head 426 of plunger 420. Accordingly, the perimeter of cap 450 is circular in shape to conform to the circular perimeter shape of electronics unit 440 and plunger head 426. Cap 450 includes a flat proximal cap surface 452 facing a proximal direction.

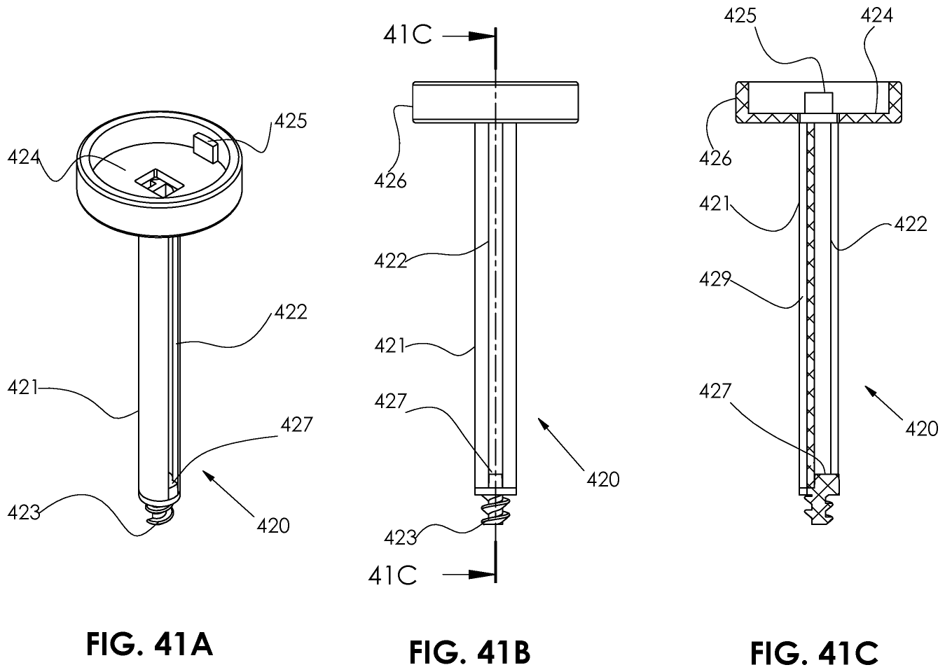

Reference is now made to FIGS. 41A, 41B, and 41C, which provide illustrative views of plunger 420 of injection device 40. Plunger 420 includes a hollow tubular plunger rod 421 aligned longitudinally. Plunger rod 421 is slidably advanceable within syringe chamber 101, such that when plunger rod 421 is pushed in a distal direction, an injectant substance contained within syringe chamber 101 is ejected distally through the aperture of syringe needle 104. A plunger rod ending 423 at the distal end of plunger rod 421 is coupled to a syringe piston 102 of syringe 100, such that the distal displacement of plunger rod 421 within syringe chamber 101 causes rod ending 423 to press against syringe piston 102 and forcing the distal displacement of the injectant substance. Plunger 420 includes a plunger head 426 at the proximal end of plunger rod 421. Plunger head 426 is configured as a receptacle with a circular disc shaped perimeter, and is configured to contain electronics unit 440 and cap 450. Plunger head 426 contains an annular surface 424 and a protrusion 425, configured to provide alignment during an assembly process, such as to facilitate alignment of components of electronics unit 420. Rack 430 is concentrically disposed within the cavity of plunger rod 421, with the distal rack surface 431 of rack 430 facing distally. Plunger rod 421 is characterized by a slot 422, extending axially on an outer surface thereof, and terminating at a distal surface 427. Plunger rod 421 is further characterized by an indentation 429 extending axially on an outer surface of plunger rod 421 opposite slot 422. Rack teeth 433 are configured to protrude out from plunger rod slot 422 when rack 430 is disposed concentrically within plunger rod 421. Ring tooth 412 of flexible ring 410 is embedded within plunger rod slot 422. Ring tooth 416 is embedded within plunger rod indentation 429, which together with ring tooth 412, prevents rotation of ring 410 relative to plunger 420, while allowing linear displacement of plunger rod 421 with respect to the ring 410. Plunger 420 is concentrically disposed within syringe 100, with ring 410 encircling plunger rod 421 at a proximal end of syringe 100.

It is noted that the functionality associated with each of the elements of injection device 40 may be distributed among multiple elements or may be performed by other elements of device 40. Injection device 40 may optionally include additional components not shown in the Figures. For example, injection device 40 may include a gripping unit with finger grippers (similar to gripping units 110, 210, 310) to facilitate depressing the plunger. Injection device 40 may also include a housing, configured to encase the device components and to allow the user to comfortably hold or grip the injection device. The injection device may further include transparent windows, configured to provide a view of the injectant substance during the injection process.

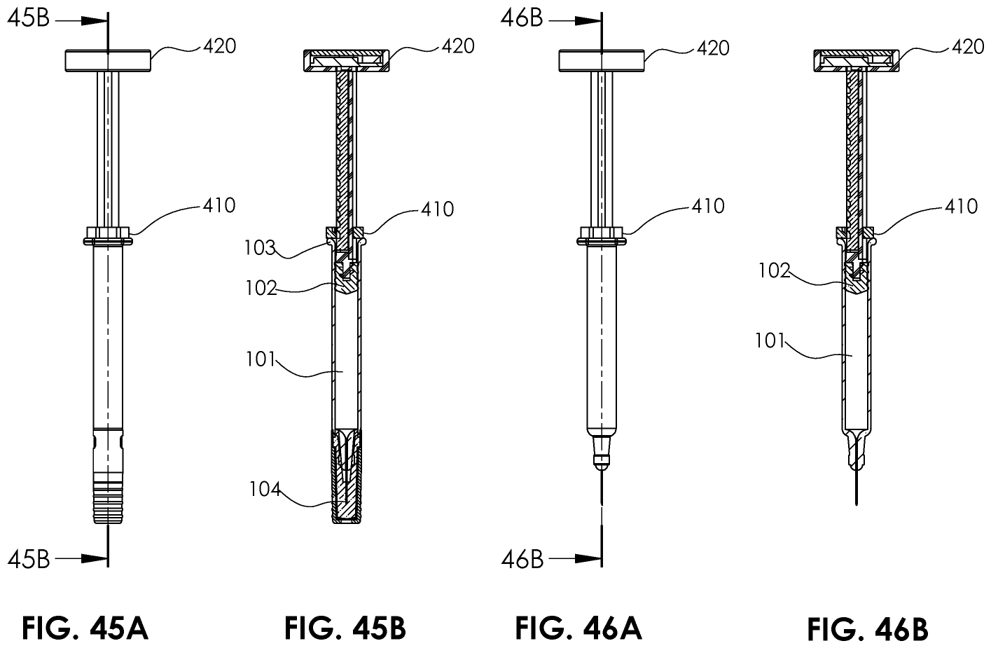

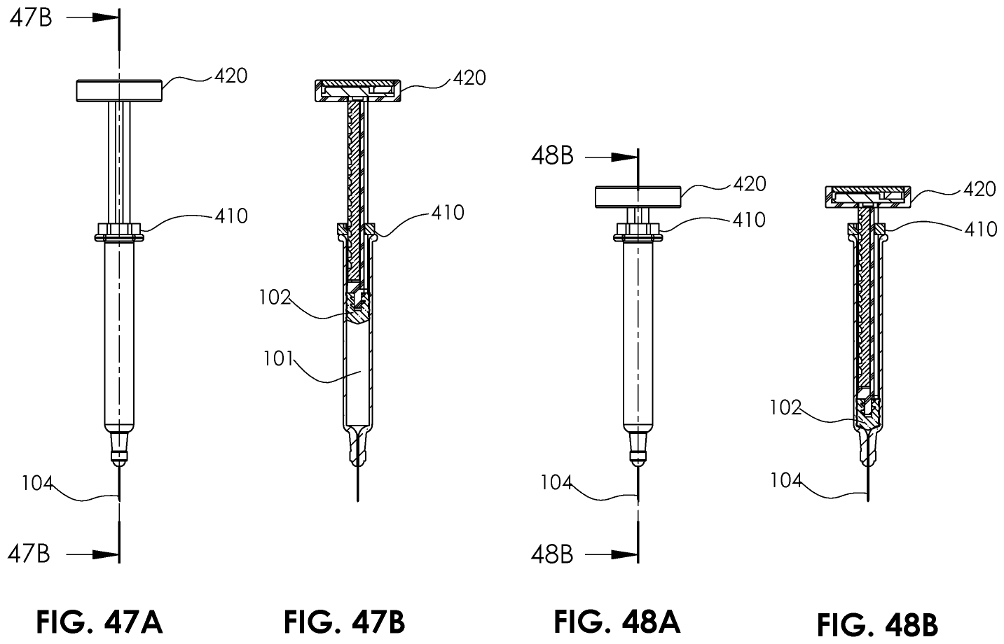

FIGS. 45A and 45B provide illustrative views of injection device 40 in an initial operational state. When injection device 40 is in a storage or non-operational state, syringe needle 104 and the distal end of syringe 100 is encased by RNS 105. To perform an injection, a user holds injection device 40, such as with fingers positioned around syringe flange 103 (or finger grippers of a gripping unit) and a thumb positioned against cap 450 at the proximal end of plunger head 426. RNS 105 is removed to expose needle 104, as shown in FIGS. 46A and 46B. The user inserts the exposed distal end of needle 104 into an injection site, such as a body area of a patient to be injected. The user then depresses plunger 420, such as by pressing distally against plunger head 426 while applying a clamping force against syringe chamber 101, causing plunger rod 421 to advance in a distal direction within syringe chamber 101. FIGS. 47A and 47B provide illustrative views of injection device 40 at a subsequent operational state with the plunger rod partially depressed. The distal advancement of plunger rod 341 propels the injectant substance distally within syringe chamber 101 to exit through the distal aperture of needle 104 and enter the injection site. FIGS. 48A and 48B provide illustrative views of injection device 40 at a later operational state with the plunger rod fully depressed.

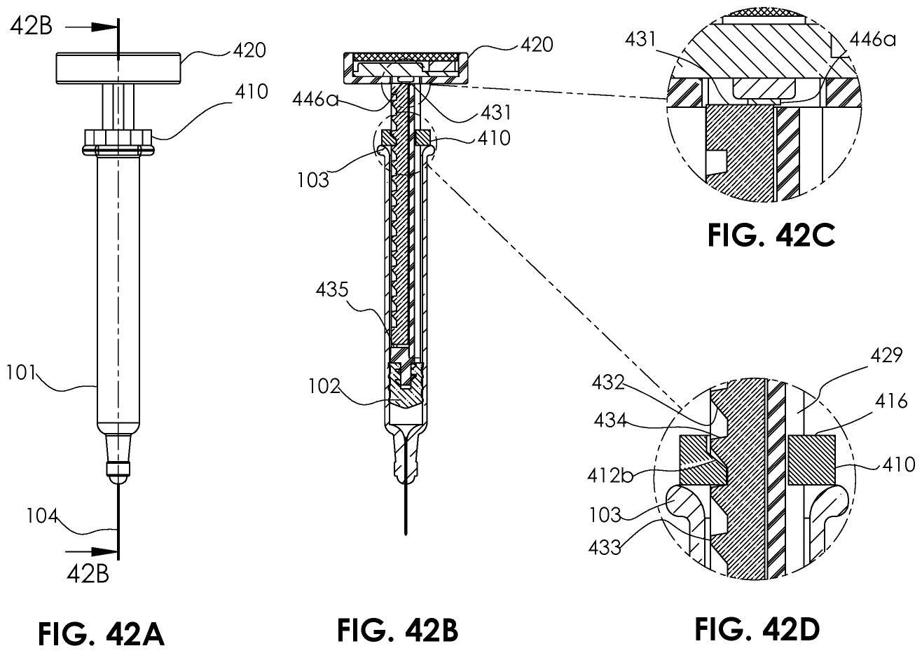

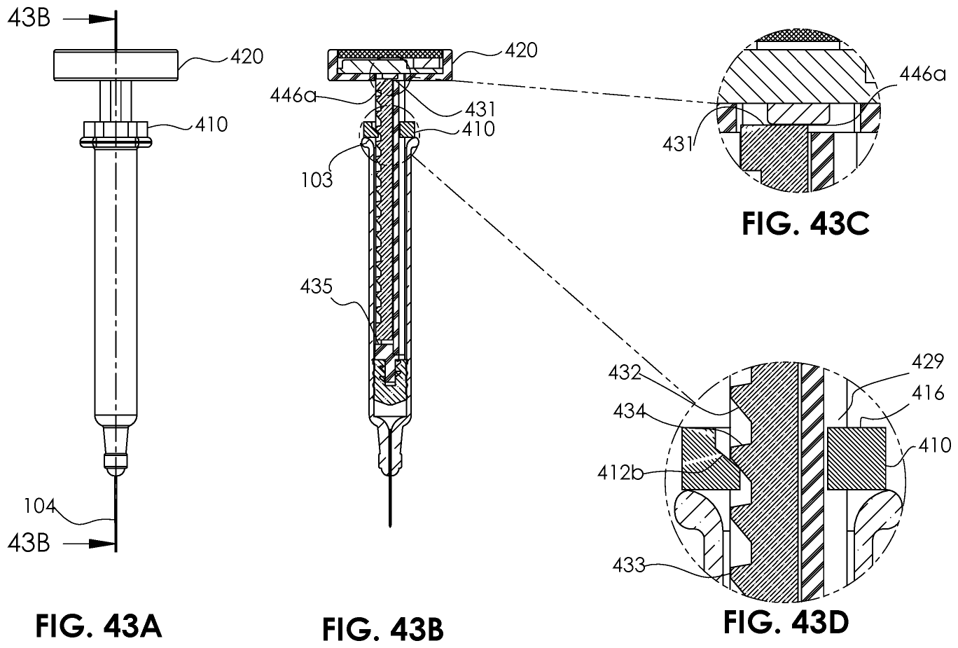

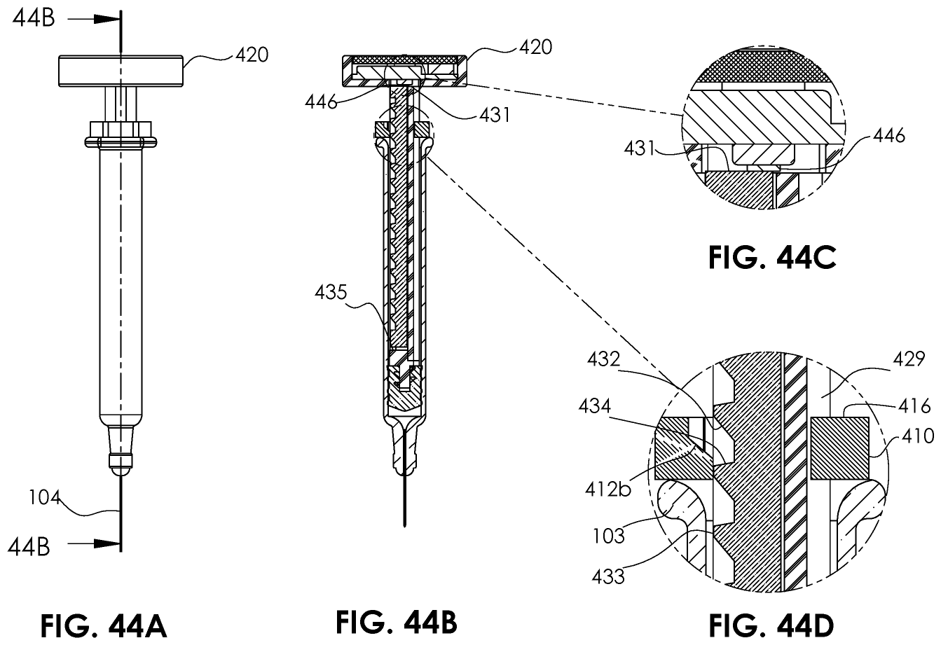

At the beginning of the injection, ring tooth 412 of flexible ring 410 engages with a rack tooth 433 of rack 430 situated within plunger rod 421. When plunger rod 421 is pressed distally, rack 430 is also pressed distally but is restricted from distal displacement by flexible ring 410, which results in a counterforce being applied by rack 430 in a proximal direction. The applied counterforce initiates the activation of sensor 446 and also initiates a radial expansion of flexible ring 410 due to the flexible nature of ring 410 and the inclined tooth faces 432 of rack teeth 433. In particular, flexible ring portions 413 bend or deform radially. Flexible ring 410 continues to expand, until rack 430 is able to advance distally by a single rack tooth 433 which pushes through ring 410, and is detected by sensor 446. FIGS. 42A, 42B, 42C and 42D provide illustrative views of injection device 40 in an initial operational state, showing sensor 446 prior to activation and flexible ring 410 prior to expansion from a proximal counterforce applied by rack 430. FIGS. 43A, 43B, 43C and 43D provide illustrative views of injection device 40 in a subsequent operational state, showing an intermediate activation stage of sensor 446, in which rack 430 is in contact with sensor 446, and an intermediate expansion stage of flexible ring 410 in which ring 410 begins to expand. FIGS. 44A, 44B, 44C and 44D provide illustrative views of injection device 40 in a yet subsequent operational state, showing a later activation stage of sensor 446 and later expansion stage of flexible ring 410. Flexible ring 410 then contracts back to its initial shape, with flexible ring portions 413 deforming back to their previous resting orientation. The aforementioned cycle then repeats itself as plunger rod 421 continues to be depressed distally, resulting in another proximal counterforce applied by rack 430, and another expansion of flexible ring 410 and corresponding distal advancement of a rack tooth 433 through flexible ring 410. Sensor 446 detects the displacement of rack 430 by detecting rack surface 435 coming into contact with sensor detection surface 446a. In particular, sensor 446 obtains a sensor reading each time a rack tooth advances through flexible ring 410, where the counterforce applied by rack 430 and initial expansion of flexible ring 410 initiates the activation of sensor 446 by the proximal rack surface 435. The cycle repeats itself for each rack tooth 433 of rack 430 until plunger rod 421 is fully depressed.

Controller 443 receives the sensor readings to generate a value representative of a linear axial displacement of plunger rod 421, which in turn may be converted to a value representative of an amount, such as a volume, of injected substance using a suitable conversion formula. The distance between adjacent rack teeth 433 determines the volume of injectant substance injected by plunger rod 421 for each sensor reading. Injection information, including an injected volume amount, along with other relevant information relating to the injection (e.g., time of injection, drug identification, expiration date, temperature), can then be transmitted via antenna 444 to a remote location, such as to a computing device (e.g., a smartphone or tablet computer) and/or a data storage unit (e.g., a cloud storage service). Alternatively, antenna 444 may transmit the detection samples directly from sensor 446 to a remote server or processor (e.g., at a cloud server), which performs the translation of the samples into linear displacement values and an injected volume amount. The final injection information may be analyzed by relevant parties to draw conclusions. For example, the injection information may be used by drug manufactures to determine manufacturing quantities for various medications or drugs, or by hospitals or medical clinic staff to determine ordering quantities for such medications, and/or by doctors or medical professionals to remotely monitor patient treatment, such as to ensure injections are properly carried out (e.g., to verify that the correct dosage amount was in fact injected, at the correct times and frequencies).

In accordance with an aspect of the present invention, at least some of the elements of injection device 40 may form a unified assembly which may be integrated with an existing syringe (e.g., a separate “add-on device”). For example, flexible ring 410, plunger 420, rack 430, electronics unit 440, and/or cap 450, may be configured as a unified device or assembly, which can be integrated with a standard syringe known in the art, such as by replacing a standard plunger and other associated syringe components.

It will be appreciated that injection devices 30 and 40 allows for remote processing and analysis of information relating to an injection process, such as quantities, times and frequencies of injection dosages. The injection information can also be monitored and analyzed to facilitate subsequent decisions regarding injections and associated treatments, such as to determine quantities of various medications to manufacture or order, and to enable remote monitoring of injections by doctors or health care personnel. For example, injection devices 30 and 40 may provide remote monitoring and verification of the injection process, by allowing verification that an injection was properly carried out, with the correct dosage amount and at a correct time and frequency.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. An injection device, the device having a distal end and a proximal end, the device comprising:
a syringe, comprising: a syringe chamber, containing an injectant substance;
a plunger, comprising: a plunger rod, axially displaceable within the syringe chamber;
at least one element, configured to undergo a corresponding element motion linked to a distal displacement of the plunger rod; and
an electronics unit, comprising at least one sensor, configured to obtain detection readings relating to the corresponding element motion of the element;
wherein an injection is performed by depressing the plunger rod in a distal direction, compelling a corresponding element motion detected by the sensor, and compelling the injectant substance to be injected through a distal end of the syringe chamber, wherein the detection readings relating to the corresponding element motion of the element is processed to determine injection information comprising at least the volume of injectant substance injected.

2. The injection device of claim 1, wherein the element comprises: a rotor, comprising a rotor rod disposed concentrically within the plunger rod; the rotor further comprising a rotor head, at a proximal end of the rotor rod,
wherein the rotor head is configured to rotate in accordance with a distal advancement of the plunger rod, such that the corresponding element motion is a rotational motion of the rotor head, and wherein the sensor is configured to detect the rotational motion of the rotor head.

3. The injection device of claim 2, wherein the rotor rod comprising a helical screw thread, and wherein the injection device further comprises at least one guiding pin, configured to engage the screw thread, so as to turn the rotor rod, producing the rotational motion of the rotor head.

4. The injection device of claim 3, wherein the guiding pin is disposed on a ring element, at least partially encircling the plunger rod, the guiding pin extending radially inward from the ring element, and wherein the guiding pin engages the screw thread through a plunger rod slot, extending axially along the plunger rod, such that the guiding pin is restricted to linear axial motion with respect to the plunger rod by the plunger rod slot, preventing rotation of the ring element with respect to the plunger rod.

5. The injection device of claim 2, wherein the rotor head comprises a plurality of rotor head apertures, and wherein the sensor is configured to detect a sequential passage of the rotor head apertures during the rotational motion of the rotor head.

6. The injection device of claim 1, wherein the element comprises: a rotor, comprising a rotor head, at least partially encircling the plunger rod,
wherein the rotor head is configured to rotate in accordance with a distal advancement of the plunger rod, such that the corresponding element motion is a rotational motion of the rotor head, and wherein the sensor is configured to detect the rotational motion of the rotor head during the distal advancement of the plunger rod.

7. The injection device of claim 6, wherein the plunger rod comprises a helical screw thread, and wherein the rotor head further comprises at least one guiding pin, extending radially inward from an inner wall of the rotor head, the guiding pin configured to engage the screw thread, so as to turn the rotor, producing the rotational motion of the rotor head.

8. The injection device of claim 6, further comprising a gripping unit, comprising a gripping unit opening, such that the plunger rod extends through the gripping unit opening, shaped with at least one edge aligned with an edge of the plunger rod, so as to prevent a rotation of the plunger rod relative to the gripping unit.

9. The injection device of claim 6, wherein the rotor head comprises a plurality of rotor head apertures, and wherein the sensor is configured to detect a sequential passage of the rotor head apertures during the rotational motion of the rotor head.

10. The injection device of claim 1, wherein the element comprises: a plurality of teeth, extending axially along a portion of the plunger rod, each tooth of the teeth comprising a respective tooth protrusion, protruding radially outwards from the plunger rod, and a respective tooth indentation, protruding radially inwards from the plunger rod,
wherein the corresponding element motion is an axial motion of the teeth, and wherein the sensor is configured to detect the sequential passage of the teeth during the distal advancement of the plunger rod.

11. The injection device of claim 10, further comprising a gripping unit, comprising a gripping unit opening, such that the plunger rod extends through the gripping unit opening, shaped with at least one edge aligned with an edge of the plunger rod, so as to prevent a rotation of the plunger rod relative to the gripping unit.

12. The injection device of claim 1, wherein the element comprises: a rack, positioned within the plunger rod, the rack comprising a plurality of teeth, extending axially along a portion of the rack, each tooth of the teeth comprising a respective tooth protrusion, projecting radially through an axial slot of the plunger rod when the rack is positioned within the plunger rod,
wherein the corresponding element motion is a sequential axial motion of the rack, and wherein the sensor is configured to detect the sequential axial motion of the rack during the distal advancement of the plunger rod.

13. The injection device of claim 12, further comprising a substantially flexible ring, at least partially encircling the plunger rod, the ring comprising at least one ring tooth extending radially inward from the ring, the ring tooth configured to engage with a rack tooth of the rack through the axial slot of the plunger rod, such that the ring is prevented from rotation with respect to the plunger rod.

14. The injection device of claim 13, wherein the ring comprises at least one flexible portion, configured to selectively deform radially, wherein during the distal advancement of the plunger rod, the rack is restricted from distal displacement by the ring tooth, resulting in a proximal counterforce applied by the rack, initiating a cyclical activation of the sensor and a cyclical radial expansion of the ring, allowing the distal advancement of a respective rack tooth through the ring, and detection of the rack tooth distal advancement by the sensor for each cycle.

15. The injection device of claim 1, wherein the electronics unit further comprises a transmitting antenna, configured to transmit the detection readings to a remote location.

16. The injection device of claim 1, wherein the electronics unit further comprises an indicator, configured to provide an indication of at least one injection state relating to the injection.

17. A method for monitoring injection information relating to an injection, the method comprising the procedures of:
providing an injection device, the device having a distal end and a proximal end, the device comprising:
a syringe, comprising: a syringe chamber, containing an injectant substance;
a plunger, comprising: a plunger rod, axially displaceable within the syringe chamber;
at least one element, configured to undergo a corresponding element motion linked to a distal displacement of the plunger rod; and
an electronics unit, comprising at least one sensor, configured to obtain detection readings relating to the corresponding element motion of the element;
performing an injection by depressing the plunger rod in a distal direction, compelling a corresponding element motion detected by the sensor, and compelling the injectant substance to be injected through a distal end of the syringe chamber; and
processing the detection readings relating to the corresponding element motion of the element to determine injection information comprising at least the volume of injectant substance injected.

18. The method of claim 17, wherein the element comprises: a rotor, comprising a rotor rod, concentrically disposed within the plunger rod, the rotor further comprising a rotor head, at a proximal end of the rotor rod,
wherein the rotor head is configured to rotate in accordance with a distal advancement of the plunger rod, such that the corresponding element motion is a rotational motion of the rotor head, and wherein the sensor is configured to detect the rotational motion of the rotor head.

19. The method of claim 18, wherein the rotor rod comprising a helical screw thread, and wherein the injection device further comprises at least one guiding pin, configured to engage the screw thread, so as to turn the rotor rod, producing the rotational motion of the rotor head.

20. The method of claim 19, wherein the guiding pin is disposed on a ring element, at least partially encircling the plunger rod, the guiding pin extending radially inward from the ring element, and wherein the guiding pin engages the screw thread through a plunger rod slot, extending axially along the plunger rod, such that the guiding pin is restricted to linear axial motion with respect to the plunger rod by the plunger rod slot, preventing rotation of the ring element with respect to the plunger rod.

21. The method of claim 18, wherein the rotor head comprises a plurality of rotor head apertures, and wherein the sensor is configured to detect a sequential passage of the rotor head apertures during the rotational motion of the rotor head.

22. The method of claim 17, wherein the element comprises: a rotor, comprising a rotor head, at least partially encircling the plunger rod,
wherein the rotor head is configured to rotate in accordance with a distal advancement of the plunger rod, such that the corresponding element motion is a rotational motion of the rotor head, and wherein the sensor is configured to detect the rotational motion of the rotor head during the distal advancement of the plunger rod.

23. The method of claim 22, wherein the plunger rod comprises a helical screw thread, and wherein the rotor head further comprises at least one guiding pin, extending radially inward from an inner wall of the rotor head, the guiding pin configured to engage the screw thread, so as to turn the rotor, producing the rotational motion of the rotor head.

24. The method of claim 23, further comprising a gripping unit, comprising a gripping unit opening, such that the plunger rod extends through the gripping unit opening, shaped with at least one edge aligned with an edge of the plunger rod, so as to prevent a rotation of the plunger rod relative to the gripping unit.

25. The method of claim 23, wherein the rotor head comprises a plurality of rotor head apertures, and wherein the sensor is configured to detect a sequential passage of the rotor head apertures during the rotational motion of the rotor head.

26. The method of claim 17, wherein the element comprises: a plurality of teeth, extending axially along a portion of the plunger rod, each tooth of the teeth comprising a respective tooth protrusion, protruding radially outwards from the plunger rod, and a respective tooth indentation, protruding radially inwards from the plunger rod,
wherein the corresponding element motion is an axial motion of the teeth, and wherein the sensor is configured to detect the sequential passage of the teeth during the distal advancement of the plunger rod.

27. The method of claim 26, further comprising a gripping unit, comprising a gripping unit opening, such that the plunger rod extends through the gripping unit opening, shaped with at least one edge aligned with an edge of the plunger rod, so as to prevent a rotation of the plunger rod relative to the gripping unit.

28. The method of claim 17, wherein the element comprises: a rack, positioned within the plunger rod, the rack comprising a plurality of teeth, extending axially along a portion of the rack, each tooth of the teeth comprising a respective tooth protrusion, projecting radially through an axial slot of the plunger rod when the rack is positioned within the plunger rod,
wherein the corresponding element motion is a sequential axial motion of the rack, and wherein the sensor is configured to detect the sequential axial motion of the rack during the distal advancement of the plunger rod.

29. The method of claim 28, further comprising a substantially flexible ring, at least partially encircling the plunger rod, the ring comprising at least one ring tooth extending radially inward from the ring, the ring tooth configured to engage with a rack tooth of the rack through the axial slot of the plunger rod, such that the ring is prevented from rotation with respect to the plunger rod.

30. The method of claim 29, wherein the ring comprises at least one flexible portion, configured to selectively deform radially, wherein during the distal advancement of the plunger rod, the rack is restricted from distal displacement by the ring tooth, resulting in a proximal counterforce applied by the rack, initiating a cyclical activation of the sensor and a cyclical radial expansion of the ring, allowing the distal advancement of a respective rack tooth through the ring, and detection of the rack tooth distal advancement by the sensor for each cycle.

31. The method of claim 17, further comprising the procedure of transmitting the detection readings to a remote location with a transmitting antenna of the electronics unit.

32. The method of claim 17, further comprising the procedure of providing an indication of at least one injection state relating to the injection, with an indicator of the electronics unit.
